SEQUENCE LISTING (6 sequences)

SEQ ID NO: 1 - protein, 326 aa, Homo sapiens
MVHVARLLLLLLTFFLRTDAETPPRFTRTPVDQTGVSGGVASFICQATGDPRPKIVWNKKGKKVSNQRFEVIEFDDGSGSVLRIQPLRTPRDEAIYECVASNNVGEISVSTRLTVLREDQIPRGFPTIDMGPQLKVVERTRTATMLCAASGNPDPEITWFKDFLPVDTSNNNGRIKQLRSESIGGTPIRGALQIEQSEESDQGKYECVATNSAGTRYSAPANLYVRELREVRRVPPRFSIPPTNHEIMPGGSVNITCVAVGSPMPYVKWMLGAEDLTPEDDMPIGRNVLELNDVRQSANYTCVAMSTLGVIEAIAQITVKALPKPP

SEQ ID NO: 2 - protein, 66 aa, Homo sapiens
IEVMYPPPYLDNEKSNGTIIHVKGKHLCPSPLFPGPSKPFWVLVVVGGVLACYSLLVTVAFIIFWV

SEQ ID NO: 3 - protein, 42 aa, Homo sapiens
KRGRKKLLYIFKQPFMRPVQTTQEEDGCSCRFPEEEEGGCEL

SEQ ID NO: 4 - protein, 112 aa, Homo sapiens
RVKFSRSADAPAYQQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPR

SEQ ID NO: 5 - RNA, 13 nt, synthetic construct
  source 1..13: mol_type = other RNA
gccgccrccatgg

SEQ ID NO: 6 - protein, 20 aa, synthetic construct
GGGGSGGGGSGGGGSGGGGS

(19) United States
(12) Patent Application Publication
(10) Pub. No.: US 2026/0241001 A1
(43) Pub. Date: Aug. 20, 2026

(54) T-CELLS COMPRISING CHIMERIC ANTIGEN RECEPTORS AND COMPOSITIONS THEREOF FOR TREATMENT OF CANCER

(71) Applicant: KING FAISAL SPECIALIST HOSPITAL AND RESEARCH CENTRE, Riyadh (SA)

(72) Inventors: Walid WARDA, Riyadh (SA); Farhatullah SYED, Riyadh (SA); Syed Osman AHMED, Riyadh (SA); Ayodele Abdulkareem ALAIYA, Riyadh (SA); Hazaa ALZAHRANI, Riyadh (SA); Hanan ALQUDAIRY, Riyadh (SA)

(73) Assignee: KING FAISAL SPECIALIST HOSPITAL AND RESEARCH CENTRE, Riyadh (SA)

(21) Appl. No.: 19/059,099

(22) Filed: Feb. 20, 2025

(51) Int. Cl. A61K 40/42; A61K 40/11; A61K 40/31; C07K 14/725; C07K 16/28; C12N 5/0783

(52) U.S. Cl. CPC A61K 40/4217; A61K 40/11; A61K 40/31; C07K 14/7051; C07K 16/2866; C12N 5/0636; C12N 2501/2307; C12N 2501/2315; C12N 2501/51; C12N 2501/515

(57) ABSTRACT

T-cells comprising chimeric antigen receptors (CARs) for the treatment of cancer. The CARs include a Protein Tyrosine Phosphatase Receptor Type D (PTPRD) domain or a single chain variable fragment of an antibody that binds to IL-1RAP expressed by cancer cells, a transmembrane domain, and domains or fragments of co-stimulatory molecules. The CAR T-cells have high specificity for IL1-1RAP expressed on the surface of cancer cells. Nucleic acids and vectors encoding the CAR T-cells and a method for production of the CAR T-cells are additional aspects.

### REFERENCE TO A SEQUENCE LISTING

In accordance with 37 CFR § 1.831-1835 and 37 CFR § 1.77 (b) (5), the specification makes reference to a Sequence Listing submitted electronically as a.xml file named “555436US_020525_ST26.xml”. The.xml file was generated on Feb. 5, 2025, and is 6,527 bytes in size. The entire contents of the Sequence Listing are hereby incorporated by reference.

### BACKGROUND

### Technical Field

The present disclosure is directed to T-cells comprising chimeric antigen receptors (CAR-T cells) specific for cancer cells expressing Interleukin-1 Receptor Accessory Protein (IL-1RAP). More particularly, the present disclosure is directed to CAR-T cells based on Protein Tyrosine Phosphatase Receptor Type D (PTPRD) that targets IL-1RAP and to a method for treating cancer using CAR-T cells.

### Description of Related Art

The “background” description provided herein is for the purpose of generally presenting the context of the disclosure. Work of the presently named inventors, to the extent it is described in this background section, as well as aspects of the description which may not otherwise qualify as prior art at the time of filing, are neither expressly nor impliedly admitted as prior art against the present invention.

AML (acute myeloid leukemia), CML (chronic myeloid leukemia), and MDS (myelodysplastic syndromes) are characterized by the dysregulated proliferation of myeloid cells, leading to ineffective hematopoiesis and severe clinical complications (See: Frenay, J. et al. IL-1RAP, a Key Therapeutic Target in Cancer. Int. J. Mol. Sci. 23, 14918 (2022)). The field of cancer treatment has long sought ways to effectively target and eliminate malignant cells while minimizing damage to healthy tissues. Despite advances in treatment, many patients experience relapse due to the persistence of leukemic stem cells (LSCs) (See: Barreyro, L. et al. Overexpression of IL-1 receptor accessory protein in stem and progenitor cells and outcome correlation in AML and MDS. Blood 120, 1290-1298 (2012)). One proposed strategy that has emerged in cancer treatment involves the targeting of Interleukin-1 Receptor Accessory Protein (IL-1RAP). IL-1RAP is a co-receptor required for signaling by the interleukin-1 (IL-1) cytokine family, and is crucial for the activation immune responses and cellular proliferation.

Overexpression of IL-1RAP in LSCs and progenitor cells has been associated with poor prognosis in these malignancies (See: R, T. et al. Chimeric antigen receptor T-cells targeting IL-IRAP: a promising new cellular immunotherapy to treat acute myeloid leukemia. J. Immunother. Cancer 10, (2022)).

IL-1RAP has also been found to be overexpressed in several types of malignant cancers, including leukemia (e.g. acute myeloid leukemia, chronic myeloid leukemia, and high-risk myelodysplastic syndrome), breast cancer, Ewing sarcoma, gastric sarcoma, colorectal cancer and pancreatic cancer, among others which suggests that it plays a role in their survival and proliferation, making it an attractive target for cancer therapy.

Overexpression of IL-1RAP may contribute to anti-apoptotic effects on cancer cells that promote their survival or that produce an inflammatory environment favorable to tumor cells.

Targeting IL-1RAP to eliminate malignant cells involves using specific molecules that can bind to IL-1RAP, thereby inhibiting its function. This inhibition disrupts the signaling pathways that are essential for the growth and survival of cancer cells, leading to their elimination. Targeting IL-1RAP also can enhance the immune system's ability to recognize and destroy malignant cells, offering a dual approach to cancer treatment. Recent advancements with this approach include the use of monoclonal antibodies that specifically bind to IL-1RAP with high affinity, blocking its interaction with IL-1, and preventing downstream signaling. Alternatively, small molecule inhibitors have been designed to interfere with IL-1RAP function, providing an alternative or concurrent therapeutic approach.

Another approach involves genetically modifying T cells to express receptors that can specifically recognize and attack cancer cells. Such receptors include chimeric antigen receptors (CARs) which are constructed by fusing a cell-surface ligand molecule with the cytoplasmic signaling domains of the receptors present on the cell surface. These chimeric receptors are designed to bind to IL-1RAP on the surface of malignant cells, triggering the immune cells to attack and destroy the targeted cancer cells. This approach leverages the natural immune response to enhance the efficacy of cancer treatment while minimizing adverse effects on healthy cells.

Accordingly, one object of the present disclosure is directed to providing T-cells comprising chimeric antigen receptors which are more specific in targeting cancer cells via interaction with IL-1RAP and hence, more effective in cancer treatment therapies.

### BRIEF DESCRIPTION OF THE INVENTION

One aspect of the invention is a chimeric antigen receptor or CAR that recognizes IL-1RAP, for example, on cancers that express or over-express IL-1RAP compared to control subjects. The CAR comprises one or more ligands for IL-1RAP. In a preferred embodiment, the ligand targeting IL-1RAP is human Protein Tyrosine Phosphatase Receptor Type D (PTPRD) or a domain or segment thereof binding to IL-1RAP. The CAR is typically expressed by a T cell and can comprise a transmembrane segment as well as internal segments that activate the CAR-T cell when the CAR binds to its target. The combination of the T cell and the CAR is designated a CAR T cell.

Another aspect of the invention is a method for treating a disease, disorder, or condition characterized or mediated by expression of IL-1RAP by administering a CAR-T cell comprising a ligand for IL-1RAP. Such conditions include, but are not limited to, cancer and inflammatory diseases.

Other aspects of the invention involve methods for targeting IL-1RAP using antibody based ligands, such as single chain variable regions, which bind to IL-1RAP. These may be used to produce CAR-T cells where an antibody ligand for IL-1RAP is used instead of a PTPRD-based ligand. Methods of targeting cells expressing IL-1RAP may also use Antibody-Dependent Cellular Cytotoxicity (ADCC) by targeting antibodies that bind to cells expressing IL-1RAP in combination with effector cells that recognize the targeting antibodies once bound to IL-1RAP on a cell.

This brief summary of the disclosure provided herein is intended to offer a concise overview and is not to be construed as limiting the scope, applicability, or configuration of the invention, which may encompass various modifications, alternatives, and equivalents without departing from the spirit and scope of the appended claims and their legal equivalents.

### BRIEF DESCRIPTION OF THE DRAWINGS

A more complete appreciation of this disclosure and many of the attendant advantages thereof will be readily obtained as the same becomes better understood by reference to the following detailed description when considered in connection with the accompanying drawings, wherein:

FIG. 1A illustrates the structure of Protein Tyrosine Phosphatase Receptor Type D (PTPRD) and Interleukin-1 Receptor Accessory Protein (IL-1RAP) complex, according to certain embodiments.

FIG. 1B illustrates the proposed interaction between PTPRD based CAR T-cell and a leukemic cell expressing IL-1RAP, according to certain embodiments.

FIG. 2 is a flowchart depicting a method for the generation of the CAR T-cell, according to certain embodiments. Reference character 100 identifies the entire series of steps and 102, 104, and 106 individual steps.

FIG. 3A illustrates the flow cytometry analysis showing IL-1RAP expression on three AML cell lines: KG-1, HL-60, and U937, according to certain embodiments.

FIG. 3B illustrates the gating strategy for flow cytometric analysis of bone marrow (BM) and blood samples, according to certain embodiments.

FIG. 3C shows IL-1RAP expression in bone marrow (BM) and blood of AML patients #1, #2 and #3.

FIG. 4A shows the schematic representation of the Lentiviral Vector Encoding PTPRD-CD28-CD32 CAR Construct, according to certain embodiments.

FIG. 4B shows CAR detection using recombinant human IL-1RAP in flow cytometry, according to certain embodiments.

FIG. 4C shows CAR protein expression evaluated by using label free quantitative liquid chromatography mass spectrometry (LC/MS), according to certain embodiments.

FIG. 5A shows the generation of T-cells, according to certain embodiments.

FIG. 5B shows a histogram showing GFP (CAR) expression, analyzed by flow cytometry, according to certain embodiments.

FIG. 5C shows the CAR vector copy number as determined by PCR on IL-1RAP CAR-T cells using MACS copy check kit, according to certain embodiments.

FIG. 6A shows the intracellular staining of TNF-α following co-culture of CAR-T cells and not transduced T cells with target cells (K562 and U937), according to certain embodiments.

FIG. 6B shows the percentage of TNF-α+ among CD3+ cells, according to certain embodiments.

FIG. 6C shows the dosage of IFN-γ concentration in co-culture media after 24 hrs. as determined by ELISA, according to certain embodiments.

FIG. 7A shows the flow cytometry analysis of CD3 expression and forward scatter in co-cultured IL-1RAP CAR-T cells with U937 (IL-1RAP-positive AML cell line) and K562 (IL-1RAP-negative control cell line) at effector-to-target ratios of 1:1, 2:1, and 4:1, according to certain embodiments.

FIG. 7B shows the quantification of target cell killing by IL-1RAP CAR-T cells, according to certain embodiments.

FIG. 8A shows the Real-Time Cell Analysis (RTCA) following co-culture of IL-1RAP CAR-T or not transduced T cells with MDA-MB-231 breast cancer cells over time, according to certain embodiments.

FIG. 8B shows the IFN-γ concentration measured by ELISA after 24 hours of coculture, according to certain embodiments.

FIG. 9 depicts some alternate embodiments of the CAR-T cell constructs.

In the drawings, like reference numerals designate identical or corresponding parts throughout the several views.

### DETAILED DESCRIPTION OF THE INVENTION

In an exemplary embodiment, a T cell comprising a chimeric antigen receptor (CAR) that comprises a ligand for Interleukin-1 Receptor Accessory Protein (IL-1RAP) is described.

PTPRD segments of CAR. In a preferred embodiment, Protein Tyrosine Phosphatase Receptor Type D (PTPRD), which is also known as Receptor-type tyrosine-protein phosphatase delta, is selected as a ligand for the IL-1RAP. One representative sequence for PTPRD is given by SEQ ID NO: 1, however, variants of PTPRD that bind to IL-1RAP are also encompassed by this term.

A variant of PTPRD may have 1, 2, 3, 4, 5, 6, 7, 8, 9, 10 or more residue deletions, substitutions, or additions to a known amino acid sequence such as SEQ ID NO: 1.

A variant of PTPRD may comprise a portion of SEQ ID NO: 1 that binds to IL-1RAP, such as a Ig2 and Ig3 domain of PTPRD.

A variant of PTPRD may have an amino acid sequence that is at least 70%, 75%, 80%, 85%, 87.5%, 90%, 92.5%, 95%, 97.5%, 98%, 99% or <100% identical or similar to SEQ ID NO: 1. It may be encoded by a polynucleotide that has 70%, 75%, 80%, 85%, 87.5%, 90%, 92.5%, 95%, 97.5%, 98%, 99% or <100% sequence identity or sequence similarity to a polynucleotide encoding the PTPRD of SEQ ID NO: 1.

Polynucleotide sequence similarity or identity may be determined using methods known in the art. These include the following: BLASTN may be used to identify a polynucleotide sequence having at least 70%, 75%, 80%, 85%, 87.5%, 90%, 92.5%, 95%, 97.5%, 98%, 99% or <100% sequence identity to a reference polynucleotide such as a polynucleotide encoding any one of SEQ ID NOS: 1-5 or other sequences disclosed herein. A representative BLASTN setting modified to find highly similar sequences uses an Expect Threshold of 10 and a Wordsize of 28, max matches in query range of 0, match/mismatch scores of 1/−2, and linear gap cost. Low complexity regions may be filtered or masked. Default settings of a Standard Nucleotide BLAST are described by and incorporated by reference to hypertext transfer protocol secure://blast.ncbi.nlm.nih.gov/Blast.cgi?PROGRAM=blastn&PAGE_TYPE-BlastSearch&LIN K_LOC-blasthome (last accessed Jan. 15, 2025).

BLASTP can be used to identify an amino acid sequence having at least 70%, 75%, 80%, 85%, 87.5%, 90%, 92.5%, 95%, 97.5%, 98%, 99% or <100% sequence identity to SEQ ID NO: 1 or other sequences disclosed herein using a similarity matrix such as BLOSUM45, BLOSUM62 or BLOSUM80 where BLOSUM45 can be used for closely related sequences, BLOSUM62 for midrange sequences, and BLOSUM80 for more distantly related sequences. Unless otherwise indicated a similarity score will be based on use of BLOSUM62. When BLASTP is used, the percent similarity is based on the BLASTP positives score and the percent sequence identity is based on the BLASTP identities score. BLASTP “Identities” shows the number and fraction of total residues in the high scoring sequence pairs which are identical; and BLASTP “Positives” shows the number and fraction of residues for which the alignment scores have positive values and which are similar to each other. Amino acid sequences having these degrees of identity or similarity or any intermediate degree of identity or similarity to the amino acid sequences disclosed herein are contemplated and encompassed by this disclosure. A representative BLASTP setting that uses an Expect Threshold of 10, a Word Size of 3, BLOSUM 62 as a matrix, and Gap Penalty of 11 (Existence) and 1 (Extension) and a conditional compositional score matrix adjustment. Other default settings for BLASTP are described by and incorporated by reference to the disclosure available at: hypertext transfer protocol secure://blast.ncbi.nlm.nih.gov/Blast.cgi?PROGRAM=blastp&PAGE_TYPE=BlastSear ch&LINK_LOC-blasthome (last accessed Jan. 15, 2025).

Variants of the other polynucleotides or polypeptides described herein by reference to a reference sequence such as one identified by a sequence identifier may be described as explained above for the PTPRD of SEQ ID NO: 1.

Polynucleotides encoding PTPRD and other peptides or proteins can be produced by those skilled in the art by reference to the genetic code. Polynucleotides may comprise a natural or degenerate sequence encoding PTPRD or other protein or peptide disclosed herein.

Linker. In addition to the portions of the CAR comprising the PTPRD ligands for IL-1RAP, a CAR as disclosed herein may comprise a hinge/spacer region which acts a flexible linker between the extracellular portion of a CAR and its intracellular domains. The linker may be derived from molecules such as (GGGGS) 4 (SEQ ID NO: 6), CD28, IgG1, IgG4, or CD8. This linker portion may serve to optimize spacing between the extracellular domain of the CAR and the T cell membrane, for example, it may extend the IL-1RAP ligand away from the T cell surface, allowing better access to the IL-1RAP on a cancer target cell.

Transmembrane domain. In some embodiments, the CAR further comprises a transmembrane domain selected from the group consisting of those of CD28, CD4 and CD8a. This domain anchors the CAR into the T cell membrane, permits signal transduction to cytoplasmic domains, and can affect CAR expression and interaction with endogenous receptors.

CD35 chain. In some embodiments, the CAR further comprises CD3 chain which participates in sign transduction following recognition of IL-1RAP.

Costimulatory domains. In some embodiments the CAR further comprises one or more costimulatory domains selected from the group consisting of CD28, 4-1BB (CD137) OX40 (CD134), ICOS (CD278), GITR (C357) and CD27. For example, CD28 and provide strong activation signals and promote rapid T cell expansion, enhanced IL-2 and other cytokine production, and provide increased effector functionality. Another example is 4-1BB (CD137) inclusion of which can improve T cell persistence, enhanced memory T cell formation and provide reduced T cell exhaustion

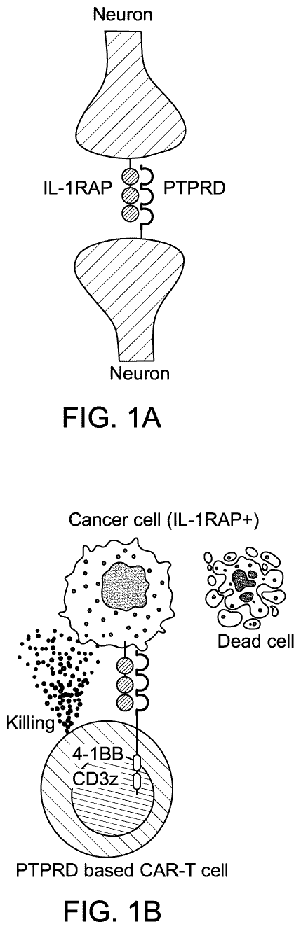

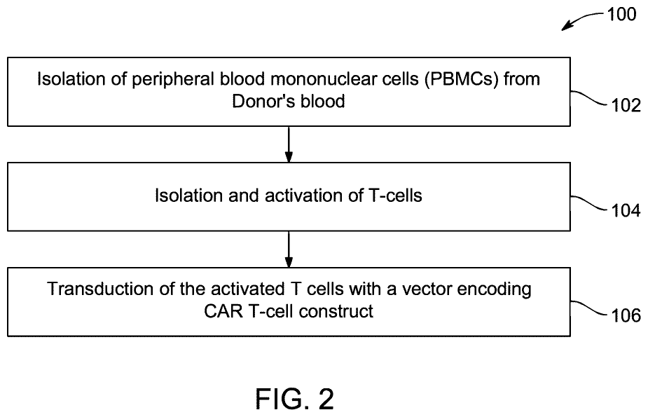

In one embodiment, the CAR-T cell comprises a PTPRD-CD28-CD3ζ CAR construct and optionally a Kozak sequence operably linked to the construct. Other embodiments comprising one or more of the specific components of CAR described in the FIGS. 1A, 4A, and 9 or as described in the Examples may be constructed.

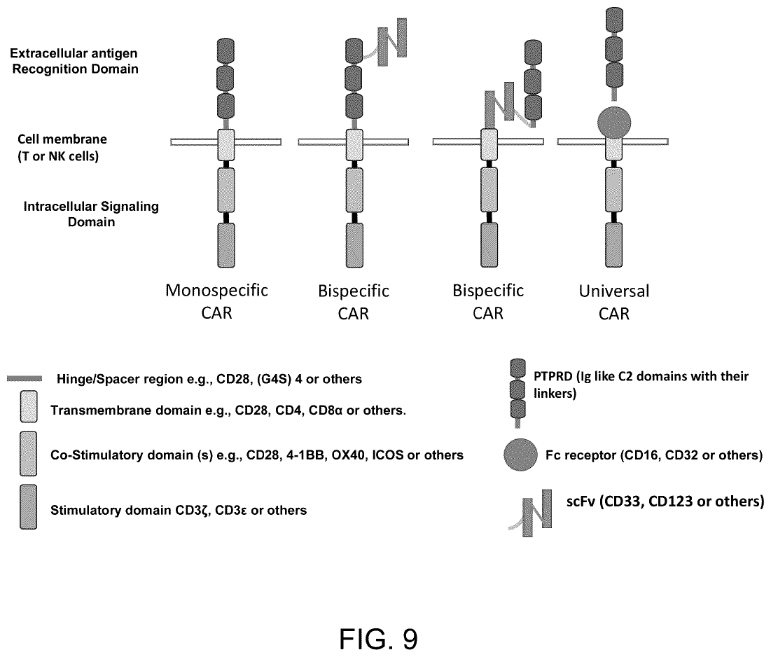

Other CAR-T embodiments comprising ligands for IL-1RAP, such as PTPRD or scFv polypeptides are also contemplated such as those illustrated in FIG. 9. A CAR may comprise one, two or more ligands for IL-1RAP. For example a ligand for IL-1RAP may comprise one or two single-chain variable fragments of an antibody that binds to IL-1RAP. Preferably, the scFv is sourced from a monoclonal antibody but those sourced from monospecific or polyclonal antibodies may be employed in some embodiments.

Cellular components of CAR T cells. In some embodiments, the T cell expressing the ligand for IL-1RAP is a CD8+ T cell, a CD4+ T cell, CD3+ T cell, or a CD56+ cell.

In some embodiments, the T cell expressing PTPRD or other ligand for IL-1RAP is a naïve T cell, a memory T cell, or a regulatory T cell.

In some embodiments, the T cell expressing the ligand for IL-1RAP is autologous to a normal cell or cancer cell expressing the IL-1RAP ligand.

In some embodiments, the T cell expressing the ligand for IL-1RAP is allogeneic to a normal or cancer cell expressing the IL-1RAP ligand, where the T cell shares at least one HLA antigen with the normal or cancer cell expressing the IL-1RAP ligand.

In some embodiments, the T cell expressing the ligand for IL-1RAP secretes one or more cytokines that damage or destroy target cells expressing IL-1RAP.

Compositions containing CAR-T cells. In some embodiments, a composition may comprise a T cell having a chimeric antigen receptor (CAR) which comprises a ligand for Interleukin-1 Receptor Accessory Protein (IL-1RAP) in combination with suitable culture medium for the CAR T cell, a CAR T cell expansion medium, a storage medium, or a medium for administration. As described below, such a composition may comprise a CAR T cells as disclosed herein in combination with a pharmaceutically or medically acceptable carrier.

Storage compositions. Such a composition may also be formulated to viably store CAR T cells as disclosed herein, for example for cryopreservation. Such a composition may comprise a cryopreservation medium, for example, when CAR-T cells are frozen or stored for later use. A cryopreservation medium may be selected by those skill in the art to preserve the viability and function of the CAR-T cells disclosed herein, for example, it may comprise saline, a protein carrier, and dimethylsulfoxide in amounts that protect the CAR-T cells during freezing and thawing. The carriers and solutions for CAR-T cells disclosed herein may be sterile, non-pyrogenic, have osmolarity close to a patient's physiological levels, and be PH-balanced (e.g.,. about pH 7.2-7.6, preferably about pH 7.4).

Infusion solutions. The CAR-T cells disclosed herein may also be suspended or incorporated into a patient infusion solution, such as a pharmaceutically acceptable saline solution (e.g., normal saline 0.9% sodium chloride), Plasma-Lyte A, Plasma-Lyte 148 or Plasma-Lyte M, and/or lactated Ringer's solution. The Plasma-Lyte family and other solutions are described by and incorporated by reference to: Rizoli, S, PMID: 21841561, DOI: 10.1097/TA.0b013e31821a4d89; Ringer's solution typically contains sodium chloride, potassium chloride, calcium chloride, and sodium bicarbonate (to buffer pH) in physiological amounts. Ringer's solution described by and incorporated by reference to hypertext transfer protocol secure://en.wikipedia.org/wiki/Ringer % 27s_solution (last accessed Jan. 14, 2025). Those skilled in the medical arts may select other suitable solutions or buffers for storage or administration of CAR-T cells these include but are not limited to Cryostor® or HypoThermosol® as well as supplementation with 5-10% Human Serum Albumin or autologous plasma to protect cells from shear stress during administration.

In some embodiments, a method for treating cancer or other condition associated with IL-1RAP or with over-expression thereof is provided. The method includes administering the T cell to a subject having the cancer or condition that expresses IL-1RAP.

Dosages. CAR-T cells are typically dosed based on the number of viable CAR+ cells per kilogram (kg) of body weight. Standard Dosage Ranges include: 1×10⁶ to 5×10⁸ CAR-T cells total or approximately 0.1×10⁶ to 10×10⁶ CAR+ cells per kg of body weight (for adults). These ranges include all intermediate values and subranges. For pediatric patients dosages are similar or are adjusted based on the patient's size and immune responses.

Dosage Adjustments: Lower doses may be used in patients with significant comorbidities or high tumor burden to minimize the risk of cytokine release syndrome (CRS) and other toxicities. Higher doses may be explored in heavily pretreated or refractory cases, provided they are well tolerated.

Frequency. CAR-T cell therapy is typically administered as a single infusion. However, depending on clinical response and trial protocols, alternative schedules may include:

Single Infusion: This is standard practice in most clinical settings.

Split-Dosing (Fractionated Infusion): Here, the total CAR-T cell dose is divided into 2-3 smaller doses over several days (e.g., Day 1, Day 3, and Day 5). This approach is used to minimize toxicity, particularly in patients with high tumor burden.

### Duration of Therapy

Initial Treatment Phase: The infusion itself typically occurs over 30-60 minutes, depending on the volume and concentration of the cell product.

Persistence of CAR-T Cells: CAR-T cells may remain active in the patient's body for months or even years, depending on the CAR design and the immune response. Second infusions are rare but may be considered if CAR-T cells fail to persist or if the disease or condition relapses.

Anatomical sites of administration of CAR-T cells. Those skilled in the art of administering CAR-T therapies may select an appropriate mode of administration and anatomical site for administration. Hematological or solid cancers and inflammatory diseases are typically treated with i.v. infusion. Solid Tumors may require intratumoral or regional administration (intraperitoneal) to overcome physical and immunosuppressive barriers. CAR-T cells also can be administered directly into the cerebrospinal fluid by intrathecal or intracerebroventricular administration.

Disease treatment. In some embodiments, the cancer or other condition comprises cancer.

In some embodiments, the cancer of other condition comprises a solid cancer.

In some embodiments, the cancer or other condition comprises a hematological malignancy or blood cancer.

In some embodiments, the cancer or other condition comprises AML (acute myeloid leukemia) or CML (chronic myeloid leukemia). Patients AML at stage M0 to M7 as determined by French-American-British (FAB) classification may be treated. Patients with CML during chronic, accelerated, or blast phase may be treated.

In some embodiments, the cancer or other condition comprises inflammatory or autoimmune disorder.

In other embodiments, patients for CAR T treatment for AML, CML or other cancers may be selected based on whether they are newly diagnosed, in remission, or having recurrent or relapsed cancer. They may be selected based on the specific type of AML, CML or other cancer, genetic or molecular characteristics of a cancer, a patient's age and overall health, white blood count at diagnosis, and response to prior treatments.

Patient factors. Patients with a high tumor burden are at greater risk of developing cytokine release syndrome (CRS) and immune effector cell-associated neurotoxicity syndrome (ICANS). These patients may require modified dosing or preconditioning to minimize toxicity. Patients with low disease burden often have better outcomes due to reduced immunosuppressive effects of the tumor microenvironment.

A “high tumor burden” in leukemia is typically quantified by a high percentage of blast cells (cancerous white blood cells) in the bone marrow, often considered to be greater than 5%; meaning a patient with a bone marrow biopsy showing more than 5% blasts would be classified as having a high tumor burden. Tumor mutational burden (TMB) used for solid cancers in binned analyses defined low TMB as ≤5, intermediate TMB>5 and <20, high TMB as ≥20 and <50, and very high TMB as ≥50 mutations/Mb with cutoffs as per prior publications; Chalmers, Z. R., et al. Genome Med. 2017, 9:34.

Pediatric Patients typically have a more robust immune system and may respond better to CAR-T cell therapy Elderly patients have increased risk of treatment-related toxicities due to comorbidities or frailty. Careful assessment is needed to ensure tolerance. A pediatric (infant) patient may be 28 days or less in age; 29 days to two years old (infant); 2 years to less than 12 years (child), or 12 through 21 years (adolescent. An elderly patient may be 60-69 years old (young-old); 70-79 years old (middle old); or at least 80 years (oldest-old).

Renal and Hepatic Function. Impaired organ function may affect the metabolism of chemotherapy used in lymphodepletion and the clearance of inflammatory cytokines.

T Cell Fitness. Patients with a history of intensive chemotherapy or stem cell transplants may have compromised T cell functionality, which could reduce CAR-T cell expansion and persistence.

Infectious Disease Status. Active infections or reactivation of latent viruses (e.g., CMV, HBV) can complicate therapy and outcomes.

Line of Therapy. Patients with relapsed/refractory disease after multiple lines of treatment may exhibit more aggressive disease or resistance to CAR T cells. A patient may have undergone 1, 2, 3 or >3 cycles of treatment such as prior exposure to immunomodulators, checkpoint inhibitors, or corticosteroids which may have altered immune responses to CAR-T therapy.

In some embodiments, the cancer or other condition comprises a proliferative disorder, an inflammatory or autoimmune disorder, a neurodegenerative disorder, or a cardiovascular disorder.

Nucleic acids and vectors. In some embodiments, a nucleic acid or nucleic acid vector encoding the CAR is provided.

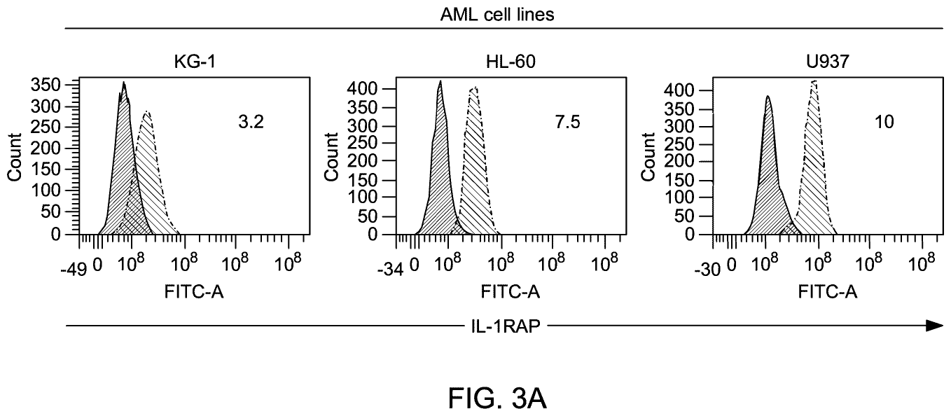

Another embodiment of the invention is directed to a method for generating the T cell. The method includes isolating PBMCs from blood, activating T cells in the PBMCs with anti-CD3/CD28 in the presence of IL-7 and IL-15 cytokines, transducing the activated T cells with a vector encoding an IL-1RAP construct, and optionally expanding the transduced T cells. A general description is provided by FIG. 2 and in the Examples. XXX

The foregoing general description of the illustrative embodiments and the following detailed description thereof are merely exemplary aspects of the teachings of this disclosure and are not restrictive.

### DETAILED DESCRIPTION OF THE INVENTION

Design and construction of the CAR-T cells disclosed herein.

As described herein, a vector for encoding a chimeric antigen receptor (CAR) comprising a ligand, like PTPRD, targeting IL-1RAP expressed by cancer cells, may include the following components.

An antigen recognition domain, such as a segment of PRPRD binding to IL-1RAP or a single-chain variable fragment of an antibody recognizing IL-1RAP. This domain would form an extracellular portion of a chimeric antigen receptor on a CAR-T cell.

A hinge/spacer region which acts as a flexible linker between the extracellular portion of a CAR and intracellular domains. This may be derived from molecules such as CD28, IgG1, or CD8. This linker portion may serve to optimize spacing between the extracellular domain of the CAR and the T cell membrane.

A transmembrane domain, which can be derived from CD28 or other membrane-spanning proteins, may be positioned to stabilize the CAR.

Intracellular signaling domains such as intracellular portions of a CD3 chain containing ITAM motifs for T cell activation.

Costimulatory domains may be included, such as one or more of those from CD28, 4-1BB, or OX40, for enhancement of T cell proliferation or persistence of T cells.

Vectors encoding CARs. Typically, a CAR construct is cloned into a viral vector, such as a lentivirus, γ-retrovirus, adeno-associated virus, CRISPR/Cas9 Genome Editing, Sleeping Beauty Transposon System, PiggyBac Transposon System, Epstein-Barr Virus (EBV)-based Vectors, Transposon/Integrase-based Systems and mRNA Electroporation which provide efficient gene transfer with or without stable integration into T cell genomes. A vector can include a strong promoter (e.g., EF1-α, CMV, PGK) to drive CAR expression. It can carry viral elements required for packaging and integration of sequences encoding the CAR as well as a selectable marker or reporter gene to identify T cells transduced with the CAR.

Production of CAR-T cells. This generally involves isolating T cells from a patient or donor, such as a patient relative or other an immunologically compatible donor. The T cells are activated using anti-CD3/CD28 beads, cytokines, or other activating molecules. The activated T cells are then transduced with the viral vector encoding the CAR and then expanded by culture in vitro. A nucleic acid encoding a CAR may be transduced into a T cell in combination with nanoparticle-based carriers, such as lipid nanoparticles comprising ionizable cationic lipids, helper lipids like phospholipids or cholesterol, or polyethylene conjugated lipids.

The T cells that have been transduced with the CAR are then selected and purified from other non-transduced cells and culture components. The anti-cancer effects of T cells transduced with a CAR may be measured and/or compared to the anticancer effects of a T cell transduced with a different CAR or other construct.

T-cells expressing chimeric antigen receptors (CARs) as disclosed herein have high specificity for Interleukin-1 Receptor Accessory Protein (IL-1RAP) present on cancer cells.

More particularly, the present disclosure relates to CARs comprising Receptor Protein Tyrosinase Phosphatase delta (PTPRD) for targeting IL-1RAP positive cells in hematological and solid tumors. A schematic illustration of the role of PTPRD in neuronal junctions and its involvement in CAR development is shown in FIG. 1A and FIG. 1B.

FIGS. 1A and 1B Schematically illustrate the normal role of PTPRD in neuronal junctions and its proposed involvement in CAR development. (1A) Structure of the PTPRD and IL-1RAP complex. The three Ig domains of PTPRD bind to the three Ig domains of IL-1RAP forming the synapse junction between two neurons. (1B) Hypothesis: Expressing a chimeric antigen derived from the Ig domains of PTPRD as the extracellular component may enable CAR-T cells to target leukemic cells expressing IL-1RAP. In FIGS. 1A and 1B: PTPRD: Receptor Protein Tyrosine Phosphatase δ, IL-1RAP: Interleukin-1 receptor accessory protein.

Receptor protein tyrosine phosphatase δ/D (PTPRδ/PTPRD) can bidirectionally induce pre- and postsynaptic differentiation of neurons by trans-synaptically binding to IL-1RAcP (See: Yamagata, A. et al. Mechanisms of splicing-dependent trans-synaptic adhesion by PTPδ-ILIRAPLI/IL-IRAcP for synaptic differentiation. Nat. Commun. 6, 6926 (2015). The junction between PTPRD and IL-1RAP is mediated by the interaction between the Ig domains of both proteins. Hence, CAR-T cells with chimeric antigen receptors comprising Ig domains of PTPRD as the extracellular element, could potentially target leukemic cells that express IL-1RAP.

Disclosed herein are T-cells expressing chimeric antigen receptors wherein the chimeric antigen receptors comprise a ligand having specificity for a surface antigen on a cancer cell. Surface antigens are protein molecules present on the surface of cancer cells resulting from mutations in the genes and consequential abnormal protein expression. Some of the surface antigens that are overexpressed on cancer cells and can be targeted for cancer therapies include Epidermal Growth Factor Receptor (EGFR), CD19, 5T4, B-Cell Maturation Antigen (BCMA), Carcinoembryonic Antigen (CEA), Interleukin-1 Receptor Accessory Protein (IL-1RAP) and the likes. IL-1RAP is a preferable target for cancer therapy as it is involved in various signaling pathways that promote cancer cell survival and proliferation. A novel splice variant of the IL-1 receptor family member IL-1RAP called IL-1RAPb was identified (See: Smith et al. A central nervous system-restricted isoform of the interleukin-1 receptor accessory protein modulates neuronal responses to interleukin-1, Immunity, 2009 Jun. 19;30 (6): 817-31). Due to conserved structural features and the tissue-specific regulation of expression. IL-1RAPb form is identical to the classic form of IL-1RAP in its extracellular and transmembrane domains and in the membrane-proximal portion of the cytoplasmic domain. Different cytokines including IL-1, IL-18, IL-33, IL-37 and IL-36 bind to IL-1RAP and promote the release of other cytokines and inflammatory mediators involved in the pathogenesis of various cancers. Accordingly, aspects of the present disclosure are directed to blocking the IL-1RAP to suppress the growth and survival of cancer cells.

In certain embodiments, the ligand is chosen based on its specificity for IL-1RAP expressed on cancer cells. In some embodiments, the ligand may be a protein molecule. In some embodiments, the entire protein molecule may be used as a ligand.

PTPRD based CARs. In specific embodiments, the ligand may be Protein Tyrosine Phosphatase Receptor Type D (PTPRD). In certain embodiments, any domain or fragment of a protein capable of binding to a surface antigen on cancer cells may be used. In specific embodiments, extracellular domains of a protein are preferred. For example, an extracellular domain of PTPRD comprising one or more Immunoglobulin (Ig)-like domains may act as a ligand. In some embodiments, the ligand comprises a domain or fragment of PTPRD having an amino acid sequence of SEQ ID NO.1. Variants having similar specificity and function as the original protein or a domain or a fragment thereof, also form a part of the present disclosure. Accordingly, in certain embodiments, the ligand comprises variants of PTPRD wherein the variants may have at least 95%, at least 96%, at least 97%, at least 98%, at least 99% identity to the amino acid sequence of SEQ ID NO: 1.

Antibody-based CARs. In some alternative embodiments, the ligand may comprise domains of an antibody capable of binding to a surface antigen or an antigen binding site on cancer cells. In specific embodiments, the present disclosure includes an antibody, a domain or a fragment thereof, as the ligand capable of binding to IL-1RAP on cancer cells. Certain domains of an antibody having high selectivity for IL-1 RAP present on cancer cells are preferable. In some embodiments, Fab fragments of an antibody having variable regions may be used. Domains derived from variable regions of either heavy or light chain of an antibody may be used. In certain embodiments, fusion proteins derived from variable regions of an antibody may be preferred. In an exemplary embodiment, the ligand is a single-chain variable fragment (ScFv) of an antibody comprising an antigen binding site for IL-1RAP. A ligand that is a single-chain variable fragment (ScFv) of an antibody is formed of variable regions of heavy and light chains of the antibody wherein the variable regions are connected by a peptide linker. The ScFv may be derived from a monoclonal antibody or a polyclonal antibody. In certain embodiments, ScFv of a monoclonal antibody is preferred.

In some embodiments, the ligand for IL-1RAP comprises a transmembrane domain for anchoring the CAR to the membrane of a T-cell. The transmembrane domain has an alpha-helical structure and may be derived from transmembrane proteins including CD3, CD8, CD28 and the likes. In some embodiments, the transmembrane domain of the CAR may be derived from proteins including IgG1, IgG4, CD32, CD28, CD4 and CD8a. The transmembrane domains may optionally, comprise one or more costimulatory molecules. Co-stimulatory molecules have intracellular signaling domains that provide secondary signals for effective activation and functioning of T-cells. The transmembrane domain of the present CARs may comprise one or more signaling domains from one or more co-stimulatory molecules.

The one or more costimulatory domains may be cytoplasmic costimulatory domains selected from a group consisting of CD3ζ chain, CD28, 4-1BB (CD137) and OX40 (CD134). In an exemplary embodiment, the ligand comprises extracellular domain of PTPRD (SEQ ID NO. 1), CD28 transmembrane region (SEQ ID NO. 2), 4-1BB (SEQ ID NO. 3) and a segment of CD3 ζ co-stimulatory molecule (SEQ ID NO. 4).

Nucleic acid sequences encoding the CARs of the present disclosure are also provided and may be deduced from the amino acid sequences described herein or are known in the art. The nucleic acid sequences can be prepared by fusion of the DNA sequence of the ligand with the DNA sequences encoding the transmembrane domain and costimulatory domains.

Vectors. The fused DNA sequence can be cloned into a vector. Any vector suitable for cloning DNA sequences may be used. In some embodiments, the vector is an expression construct capable of delivering the desired gene into the host cell. In some embodiments, the vector is a plasmid. In other embodiments, the vector is a linear nucleic acid. The linear nucleic acid may a linear DNA or RNA. Suitable vectors that can be used for cloning the CAR nucleic acid sequence comprise viral vectors including lentiviral, adenoviral or adeno-associated viral vectors. In a specific embodiment, the CAR sequence is cloned into a lentiviral vector.

In another specific embodiment, the CAR sequence of the present disclosure is cloned into an HIV based lentiviral vector. In an exemplary embodiment, a Lentiviral Vector Encoding PTPRD-CD28-4-1BB-CD3ζ CAR Construct is provided.

In some embodiments, the CAR construct may include additional nucleic acid motifs for efficient translation initiation. For example, a Kozak sequence (may be operably linked to the PTPRD-CD28-CD3 CAR construct to achieve efficient translation of the DNA sequence into protein.

The T-cells comprising the CAR of the present disclosure may be cytotoxic T-cells (CD8+) or helper T-cells (CD4+). Cytotoxic T-cells show cytolytic activity and are capable of directly killing the target cells. Helper T-cells assist other immune cells by releasing cytokines that activate cytotoxic T cells, B cells, and other immune cells like macrophages. In some embodiments, the T-cell may be a naive cell. In other embodiments, the T-cell may be a memory T-cell. In some other embodiments, the T-cell may be a regulatory T-cell. In certain embodiments, the T-cells may be autologous T-cells expressing IL-1RAP. In such cases, the T-cells of a normal person or from a person requiring cancer therapy may be modified to express the CARs of the present disclosure. Alternatively, the T-cells may be allogeneic to a normal cell or a cancer cell expressing IL-1RAP. However, to limit HLA incompatibility in such cases, the allogeneic T-cell must have at least one human leukocyte antigen (HLA) in common with the HLAs on a normal or cancer cell expressing the IL-1RAP.

In some embodiments, the CAR-T cells release one or more cytokines to trigger the cytotoxic activity of the T-cells. The cytokines may be released during immunological synapse and may include various interleukins like IL-2, IL-4, IL-5, IL-9, IL-12, IL-13, IL-17a, IL-17f, IL-22, tumor necrosis factor-alpha (TNF-α) and interferon-gamma (IFN-γ). The secretion of one or more of these cytokines damages and/or destroys cancer cells expressing IL-1RAP.

Selection of T cells transduced with CAR. One skilled in the art may select a method for purifying or isolating CAR transduced T cells. Such methods include antibiotic selection based on resistance markers in the transducing vector; flow cytometry based cell sorting; magnetic beads-based selection, e.g., using biotinylated antibodies binding to specific CAR components and streptavidin-coated magnetic beads; separation of transduced cells by density gradient centrifugation; or by buoyancy activated separation. Automated systems may be employed to isolate CAR T cells, such as the Gibco CTS DynaCellect® Magnetic Separation System (Thermo Fischer Scientific).

Compositions comprising CAR-T cells and one or more pharmaceutically acceptable carrier or excipients also form a part of the present disclosure. Suitable excipients may include one or more lipids, salts, preservatives, stabilizers and sugars. Cationic or non-cationic lipids may be useful in the formulation of said compositions. Suitable cationic and non-cationic lipids may be those described in the prior art. For example, ionizable lipids, polyethylene glycol lipids, polyamide oligomers and the likes. Salts including calcium phosphates, aluminium salts including aluminium hydroxide, aluminium phosphate, sodium salts, potassium salts may be added to the composition for faster and efficient delivery to the subject. Phosphate buffered saline (PBS) may be added to the compositions to maintain the structure, form and shape of the cells The compositions may further comprise stabilizers like gelatin, polysorbates, preservatives like thimerosal and the likes. Sugars including sucrose, glucose and lactose may also be added to the compositions as they act as preservatives.

One embodiment of the method for generating CAR T cells is shown by FIG. 2, reference character 100. A schematic illustration of the method 100 is shown in FIG. 2. At step 102, the method 100 includes isolation of peripheral blood mononuclear cells (PBMCs) from the blood of a donor. The step 102 includes drawing a blood sample from the donor and subjecting them to density gradient centrifugation. The centrifugation process separates various components of the blood based on their density, allowing for the precise extraction of PBMCs. PBMCs may include various lymphocytes such as T cells, B cells, and NK cells and monocytes. T-cells from the PBMCs are then isolated using magnetic-activated cell sorting (MACS) wherein CD4/CD8-specific magnetic beads coated with antibodies specific for CD4 and CD8 molecules on the surface of the T cells, are used to isolate CD4+ and CD8+ T-cells.

At step 104, the method 100 includes activation of the isolated T-cells. The isolated CD4+ and CD8+ T-cells are cultivated in a cell culture medium preferably RPMI medium and activated using anti CD3/CD28 antibodies. The anti CD3/CD28 antibodies act as stimulators for T-cell activation. Additionally, cytokines including IL-7 and IL-15 are added to the T-cell activation medium that help in the proliferation and survival of the activated T cells.

At step 106, the method 100 includes subjecting the activated T-cells to transduction with a vector wherein the activated T-cells are transduced with a vector encoding the CAR-Lentiviral construct. The transduction is carried out for at least 24 hours while the activated T-cells are in the cell culture medium and can be repeated two or more times if needed. The transduced T-cells express the CAR of the present disclosure on their surface.

Optionally, the method 100 includes expansion of CAR T-cells obtained in step 106. The CAR T-cells are cultivated in the RPMI medium for at least 8-10 days under conditions desirable for the expansion of the T-cells. In some embodiments, the T-cells are cultivated for at least 9 days to obtain a sufficient number of transduced T-cells. The CAR T-cells are then evaluated for the transduction efficiency by flow cytometry using anti-CD3 antibodies.

Therapy. The CAR T-cells of the present disclosure can be used alone or in combination with other therapies for the treatment of cancers. The CAR T-cells can also be used to prevent relapse of cancer following an initial treatment, thereby ensuring a long-term remission from cancer. The compositions comprising CAR T-cells can be administered by parenteral, enteral or topical modes. Parenteral mode of administration may include intravenous, intramuscular, subcutaneous, intradermal, intraperitoneal, intrathecal, intraarterial, intracardiac modes and the likes. In certain cases, the compositions may be administered via oral, sublingual, buccal and rectal routes. Topical modes of administration may be preferred if other modes of administration are not possible.

Cancer types. The compositions of the present disclosure can be used for the treatment of solid cancers and malignant cancers expressing IL-1RAP. The type of cancer may comprise solid tumors including sarcomas, carcinomas, and lymphomas. For example, the solid cancer may be breast cancer, ovarian cancer, esophageal cancer, retinoblastoma, lung cancer or neuroblastoma. The cancer may be a hematological malignancy or blood cancer including myeloblastic leukemia, acute and chronic myeloblastic leukemia. The CAR T-cells of the present disclosure may also be used for the treatment of other conditions characterized by proliferative disorder, an inflammatory or autoimmune disorder like rheumatoid arthritis, a neurodegenerative disorder, or a cardiovascular disorder such as atherosclerosis wherein an active role of IL-1RAP in the progression of the disease or disorder is observed.

### EXAMPLES

The following examples demonstrate the preparation of CAR T-cells as described herein. The examples are provided solely for illustration and are not to be construed as limitations of the present disclosure, as many variations thereof are possible without departing from the spirit and scope of the present disclosure.

### Example 1

### Design and Production of PTPRD Derived Peptide CAR Plasmid

Amino-acids sequence of PTPRD, CD28, 4-1BB human proteins were identified using online database (worldwide web.uniprot.org). Exemplary polypeptide sequences for producing a CAR are described below. Other functionally and structurally similar sequences may also be used, for example, a sequence having at least 70, 80, 90, 95, 99, or <100 sequence identity to a sequence described below that retains its essential function within a CAR.

(1) The Ig domains (Ig1, Ig2 and Ig3) of human PTPRD protein (UniProt ID: P23468, incorporated by reference) with the sequences of signal peptide and linear peptidic epitopes are described below:

(SEQ ID NO. 1)
MVHVARLLLLLLTFFLRTDAETPPRFTRTPVDQTGVSGGVASFI
CQATGDPRPKIVWNKKGKKVSNQRFEVIEFDDGSGSVLRIQPLRT
PRDEAIYECVASNNVGEISVSTRLTVLREDQIPRGFPTIDMGPQLK
VVERTRTATMLCAASGNPDPEITWFKDFLPVDTSNNNGRIKQLRS
ESIGGTPIRGALQIEQSEESDQGKYECVATNSAGTRYSAPANLYVR
ELREVRRVPPRFSIPPTNHEIMPGGSVNITCVAVGSPMPYVKWML
GAEDLTPEDDMPIGRNVLELNDVRQSANYTCVAMSTLGVIEAIAQ
ITVKALPKPP.

The initial underlined sequence above is a signal peptide sequence. The second third, and fourth underlined sequences are linkers. The intervening not underlined sequences comprise Ig1, Ig2 and Ig3 domains of PRPRD.

The topological (extracellular) domain of human PRPRD comprises positions 21-1265, the transmembrane (helical) domain comprises positions 1266-1290, and the topological (cytoplasmic) domain comprises positions 1291-1912.

Representative sequences follow.

The signal peptide sequence

(SEQ ID NO: 7)
MVHVARLLLLLLTFFLRTDA
Ig like C2-type 1 (24-114)
(SEQ ID NO: 8)
PRFTRTPVDQTGVSGGVASFICQATGDPRPKIVWNKKGKKVSNQRFEVIE
FDDGSGSVLRIQPLRTPRDEAIYECVASNNVGEISVSTRLT
Ig-like C2-type 2 (126-224)
(SEQ ID NO: 9)
PTIDMGPQLKVVERTRTATMLCAASGNPDPEITWFKDFLPVDTSN
NNGRIKQLRSESIGGTPIRGALQIEQSEESDQGKYECVATNSAGTRY
SAPANLY
Ig-like C2-type 3 (236-318)
(SEQ ID NO: 10)
PRFSIPPTNHEIMPGGSVNITCVAVGSPMPYVKWMLGAEDLTPEDD
MPIGRNVLELNDVRQSANYTCVAMSTLGVIEAIAQIT.

A CAR may comprise 1, 2, or 3 Ig-like C2 domains of PTPRD.

(2) A part of extracellular sequence and the transmembrane sequence of human CD28 (UniProt ID: P10747, incorporated by reference):

(SEQ ID NO. 2)
IEVMYPPPYLDNEKSNGTIIHVKGKHLCPSPLFPGPSKPFWVLVVV
GGVLACYSLLVTVAFIIFWV.

The topological (extracellular) domain of human CD28 comprises positions 19-152, the transmembrane (helical) domain comprises positions 153-179, and the topological (cytoplasmic) domain comprises positions 18-220.

(3) The intracellular region of 4-1BB (ID: Q07011, incorporated by reference):

(SEQ ID NO. 3)
KRGRKKLLYIFKQPFMRPVQTTQEEDGCSCRFPEEEEGGCEL

The topological (extracellular) domain of human TNR9 comprises positions 24-186, the transmembrane (helical) domain comprises positions 187-213, and the topological (cytoplasmic) domain comprises positions 214-255.

TNR9_HUMAN is a gene that codes for the 4-1BB protein, also known as CD137, which belongs to the tumor necrosis factor receptor (TNFR) superfamily.

(4) A segment from the intracellular fragment of human of CD3z (ID: P20963, incorporated by reference):

(SEQ ID NO. 4)
RVKFSRSADAPAYQQGQNQLYNELNLGRREEYDVLDKRRGRDPE
MGGKPRRKNPQEGLYNELQKDKMAEAYSEIGMKGERRRGKGHD
GLYQGLSTATKDTYDALHMQALPPR.

The topological (extracellular) domain of human CD3Z comprises positions 22-30, the transmembrane (helical) domain comprises positions 31-51, and the topological (cytoplasmic) domain comprises positions 52-164.

(5) A “Kozak sequence” which refers to (gcc) gccRccAUGG (SEQ ID NO: 5), where R is a purine (adenine or guanine). This sequence is typically positioned three bases upstream of the start codon (AUG) and is followed by another “G”.

Variants or fragments of the above molecules of SEQ ID NOS: 1-10 or the other sequences described herein that retain functionality may be used for producing a CAR as disclosed herein. Such variants may have 90, 91, 92, 93, 94, 95, 96, 97, 98, 99 or <100% sequence identity with SEQ ID NOS: 1-10 or contain 1, 2, 3, 4, 5, 6, 7, 8, 9, 10 or >10 deletions, substitutions or insertions of amino acid residues.

In Example 1, next the DNA sequence encoding of selected PTPRD oligo-peptide, CD28, 4-1BB and CD3z were obtained by corresponding codon using online tools (www.bioinformatics.org). Then, a codon optimization for mammalian expression has been done before the DNA synthesis of all CAR segments (in order: PTPRD derived oligo-peptide, CD28 hinge and transmembrane segment and CD3z segment) by Synbio-technologies (New Jersey, USA). Next, CAR DNA sequence was cloned in the multiple cloning site of a lentiviral backbone pCDH-EF1-MCS-CMV-copGFP-T2A-Puro.

Cells. CAR-T cells were generated by transducing primary human T cells with a lentiviral vector encoding the CAR construct, followed by expansion in culture. Not transduced T cells were maintained under identical conditions. K562 and U937 cell lines were cultured in RPMI-1640 medium supplemented with 10% fetal bovine serum (FBS) and 1% penicillin-streptomycin.

CAR-T cell production. Blood samples from healthy donors were collected and obtained after written informed consent (RAC #2240004, KFSH&RC, Riyadh). On day 0 (DO), PBMCs will be separated from other cells by density gradient centrifugation using Ficoll (Sigma). Then, CD4+ and CD8+ cells will be magnetically isolated with CD4/CD8 beads (Miltenyi™). A total of 10×10⁶ cells were activated using T cell TransAct™ (Miltenyi), a colloidal polymeric nanomatrix covalently attached to humanized recombinant agonists of CD3 and CD28. Twenty-four hours after T cell stimulation, CAR lentiviral supernatant will be added. In parallel, control cells (not transduced T cells) were produced also. Subsequently, cells were expanded for 9 days. During the entire process, T cells will be cultivated in RPMI medium (Gibco) supplemented with the recombinant human cytokines IL-7 (1700 UI/mL) and IL-15 (320 UI/mL) (Milteny™). On day 9, the transduction efficiency was evaluated by flow cytometry using anti-CD3. Over the entire process, quality control tests enabled in-process assessment of cell count and viability via trypan blue.

IL-1RAP expression in flow cytometry: Peripheral blood mononuclear cells (PBMCs) were isolated from AML patient samples using density gradient centrifugation with Ficoll-Paque PLUS. Briefly, peripheral blood was collected in EDTA-coated tubes. The blood was carefully layered over Ficoll-Paque PLUS and centrifuged at 400×g for 30 minutes at room temperature without brake. The PBMC layer was collected from the plasma-Ficoll interface, transferred to a new tube, and washed twice with PBS to remove platelets and residual Ficoll. The final PBMC pellet was resuspended in PBS. For flow cytometry analysis, 1×10⁶ PBMCs were stained with anti-human CD45-pacific blue (Miltenyi), anti-human CD33-PE (BD Biosciences, xx), and anti-human IL-1RAP-FITC (R&D systems) antibodies. Cells were incubated with the antibodies for 15 minutes at 4° C. in the dark, followed by a wash with PBS. To exclude dead cells, 7-AAD viability staining was performed for 10 minutes before analysis. Stained cells were analyzed on a BD LSRII flow cytometer, and data were acquired for at least 10,000 events per sample. Compensation was performed using single-stained controls, and data were analyzed with FACS DIVA software. The leukocyte population was gated on CD45+ cells, and within this gate, the CD14-blast population was identified. IL-1RAP expression was assessed within the CD45low/CD14-gate, and the mean fluorescence intensity (MFI) was calculated.

IL-1RAP CAR expression in flow cytometry: HEK293T cells were transduced with IL-1RAP CAR vector. Cells were then incubated in presence of recombinant human IL-1RAP conjugated to human Fc (Sino biological, 10121-H02H). Staining was performed with Alexa Fluor 647 labeled anti-human Fc antibody (Invitrogen, A21445) and analyzed by flow cytometry (LSR Fortessa, BD Biosciences). Wild type HEK293T cells were used as negative control.

TNF-α intracellular staining: Effector cells (untransduced T cells and CAR-T cells) were co-cultured with either K562 or U937 cells at a E: T ratio of 1:4 for 6 hours in presence of Golgi Plug (BD Bioscience). Effector cells alone were used as negative control. At the end of co-culture, cells were stained with anti-CD3 FITC. After washing, cells were fixed/permeabilized using BD Cytofix/Cytoperm Kit (554714) and cells were stained with anti-TNF-α PE-Cy7. Data were acquired on an LSRII flow cytometer (BD Biosciences). BD FACSDiva software (BD Biosciences) was used for further data analysis.

Cell lysis and fractionation: Cell lysis and fractionation were conducted to isolate cytoplasmic and nuclear fractions from wild-type HEK293T cells and CAR-transduced HEK293T cells using the Subcellular Protein Fractionation Kit for Cultured Cells (ThermoFisher, #78840). Cells were harvested and washed twice for 3 minutes at 500×g with cold phosphate-buffered saline (PBS) to remove any residual media. Cells were washed with PBS and pelleted. The cell pellet was resuspended in 1 mL of Buffer 1 and incubated for 10 minutes on an end-over-end shaker. The lysate was then centrifuged at 500×g for 5 minutes. The resulting supernatant, containing the cytosolic proteins, was collected. The remaining pellet was resuspended in 1 mL of Buffer 2, vortexed, and incubated for 10 minutes on an end-over-end shaker. After centrifugation at 3,000×g for 5 minutes, the supernatant containing primarily membrane proteins was collected. The pellet was then dissolved in 0.5 mL of Buffer 3, vortexed, and incubated for 30 minutes on an end-over-end shaker. Following centrifugation at 5,000×g for 5 minutes, the supernatant containing soluble nuclear proteins was collected. Buffer 4 was prepared at room temperature by adding 25 μL of 100 mM CaCl₂) and 15 μL of micrococcal nuclease to 0.5 mL of Buffer 3. This buffer mixture was added to the remaining cell pellet, vortexed, and incubated for 15 minutes at room temperature. The mixture was vortexed for 15 seconds and centrifuged at 16,000×g for 5 minutes, resulting in a supernatant containing chromatin-bound nuclear proteins. The final pellet was resuspended in 0.5 mL of Buffer 5, vortexed, and incubated for 10 minutes at room temperature. After centrifugation at 16,000×g for 5 minutes, the resulting supernatant containing cytoskeletal proteins was collected. Protein content in all fractions was determined using the BCA Protein Assay Macro Kit according to the manufacturer's instructions.

Proteomics analysis using label free quantitative liquid chromatography mass spectrometry (LC/MS): All samples were subjected to subcellular protein fractionation using the Thermo Fisher Kit, (Cat: 78840). Approx. 200-400 μl extraction fractions from different samples were available for analysis. All samples were reduced to 200 μl using the refrigerated speed vacuum concentrator and protein concentration determined for each sample. Equal amount (100 μg) of complex extracted proteins mixture from each sample groups were subjected to in solution tryptic digestion. Briefly, all samples, were heat denatured at 80° C. for 15 min, follow by reduction in 10 mM DTT at 60° C. for 30 min and subsequently alkylated using 50 mM Iodoacetamide, in the dark for 40 minutes at room temperature. All samples were trypsin digested at a ratio of 1:30 (w/w) (Promega, sequence grade trypsin) at 37° C. for 12 hrs. The extracted peptides were subjected to mass spectrometry analysis using label-free quantitative Nano Acquity liquid chromatography coupled to Synapt G2. Data was acquired using the ion mobility separation HDMSE on a Trizaic Nano-flow source (Waters, Manchester, UK). The MS data were obtained from a range of m/z 50-2000 Da using 2 hours' gradient acquisition run time and all samples were as analyzed in triplicate runs using the Mass Lynx platform (Version. 4.1, SCN833).

Bioinformatics and Data Analysis: Progenesis QI for proteomics (QIfP) V 3.0 (Waters/Nonlinear Dynamics, UK) was used for all computer assisted data processing and database searching. The observed qualitative differentially expressed global proteins between sample groups were identified. Briefly, the acquired list of peptide ions for protein identification will be queried in unified UniProt/SwissProt non-redundant human protein sequence database, (www.uniprot.org) in which the PTPRD based CAR partial sequence was added. The list of all identified protein datasets was further reviewed for the presence or absence of the PTPRD based CAR partial sequence in HEK293T transduced and untransduced samples based on the normalized label-free qualitative and quantification expression values.

ELISA: CAR-T cells were co-cultured with K562 (IL-1RAP-negative cell line) or U937 (IL-1RAP-positive AML cell line) to evaluate their functional activity, with untransduced T cells serving as a negative control and a stimulation cocktail as a positive control. Co-cultures were set up in 96-well U-bottom plates at an effector-to-target (E) ratio of 1:4, with 1×10⁵ effector cells and 4×10⁵ target cells per well, in a total volume of 200 μL. Negative control wells contained untransduced T cells co-cultured with K562 or U937 cells, while positive control wells contained CAR-T cells stimulated with a stimulation cocktail (ref) in the absence of target cells. All conditions were set up in triplicate and incubated at 37° C. with 5% CO₂ for 24 hours. After incubation, supernatants were harvested by centrifugation at 300×g for 5 minutes, and the resulting supernatants were collected for analysis. IFN-γ levels in the supernatants were quantified using a human IFN-γ ELISA kit according to the manufacturer's instructions. Briefly, 100 μL of each supernatant was transferred to the ELISA plate, followed by incubation with detection antibodies and substrate solution. Absorbance was measured at 450 nm using an ELISA plate reader, and IFN-γ concentrations were calculated based on a standard curve generated using recombinant IFN-γ standards.

Vector Copy Number: The vector copy number (VCN) assay is intended to determine the average copies of integrated lentiviral vectors per cell genome after ex vivo transduction of human cells. After isolating genomic DNA from the transduced cells using QIAamp DNA Mini Kit t (Qiagen, 51304), the copies of the lentiviral gag gene are determined in relation to the human reference gene PTBP2 by quantitative PCR. The MACS COPYcheck Kit (Miltenyi Biotec, 130-128-157) provides specific primers to amplify in real time quantitative PCR an amplicon of the lentiviral gag gene and the human reference gene PTBP2, respectively. Copy number per transduced cell=qPCR copy number/μL (gag)+qPCR copy number/μL (PTBP2)/Transduction frequency (%)=100%×2.

CAR-T cell killing assay against AML cells: Cytotoxicity assays were performed by co-culturing CAR-T cells with target cells at various effector to target ratios. Briefly, 100,000 target cells (K562 or U937) were plated per well of a 96-well plate and various ratios of CAR-T cells were added. At the end of the culture, cells were stained with DAPI for viability, CD3 for the identification of the effector cells. After 15 min, Flow cytometry Absolute count standard (Thermo Fisher) were added to the tube and cells were analyzed by flow cytometry. Absolute numbers of live target cells at the end of the assay were calculated by comparing the ratio of bead events to cell events. The percentage of cytotoxic activity was determined by dividing the absolute number of target cells at the end of the culture with the absolute number of target cells at the end of the experiment obtained with untransduced T cells.

Statistical Analysis: All statistical analyses were done using GraphPad Prism 8.3.0 (GraphPad Software, San Diego, CA, United States). The results were presented as mean standard deviation (SD) of triplicate wells, and statistical significance was evaluated using a two-tailed unpaired t-test, with a p-value <0.05 considered statistically significant. Statistical significance was given as *, **, *** by p-values less than <0.05, <0.001 or <0.0001, respectively.

### Results

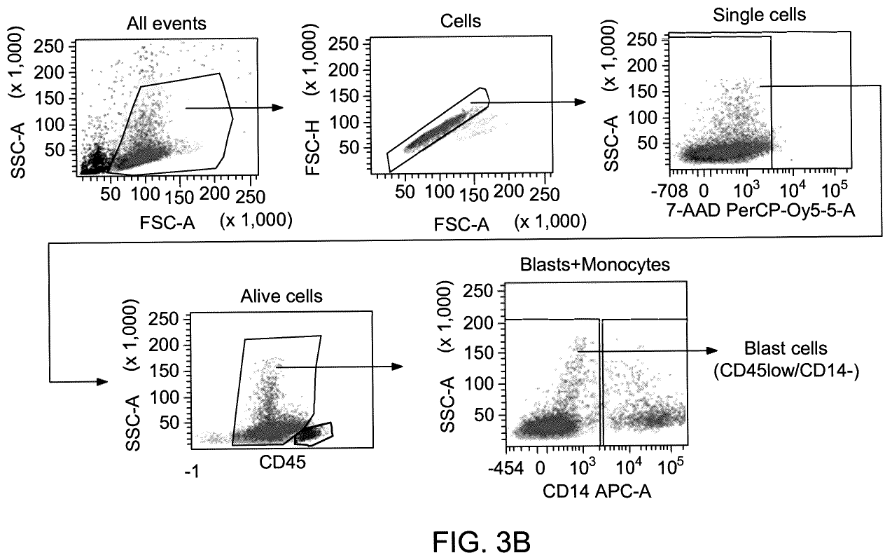

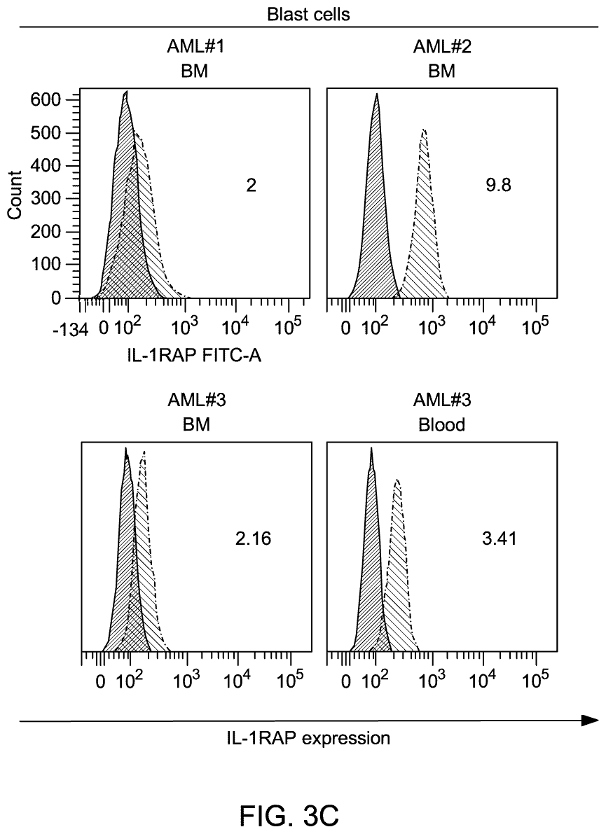

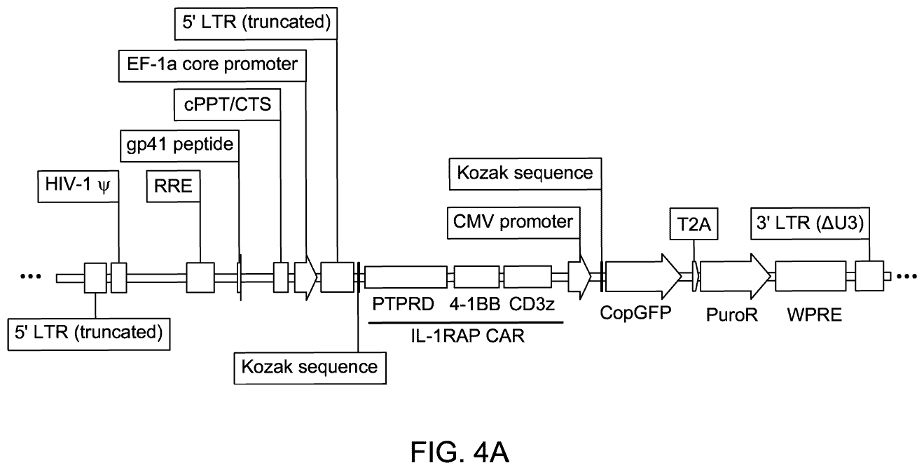

IL-1RAP antigen is expressed on the surface of AML cells. Il-1RAP expression level was determined by flow cytometry using a mouse monoclonal antibody. It was demonstrated that both AML cell line (KG-1, HL-60 and U937) and AML primary cells (4 patients) obtained from either blood or bone marrow express IL-1RAP on their surface (FIG. 3A to FIG. 3C).

FIGS. 3A-3C show the expression of IL-1RAP on AML cell lines and patient-derived samples. A) Flow cytometry analysis showing IL-1RAP expression on three AML cell lines: KG-1, HL-60, and U937. The green histograms represent IL-1RAP-positive cells, and the black histograms represent isotype controls. The ratio of mean fluorescence intensity values of IL-1RAP are shown for each cell line. B) Gating strategy for flow cytometric analysis of bone marrow (BM) and blood samples. Sequential gating of viable cells based on forward scatter (FSC-A), side scatter (SSC-A), 7-AAD viability dye, CD45, and CD14 expression is demonstrated. This gating identifies leukocyte populations for further IL-1RAP analysis. Flow cytometry histograms represent IL-1RAP-positive (red) and isotype control (blue) cells. BM: Bone marrow. The ratio of mean fluorescence intensity values of IL-IRAP are shown for each AML patient's sample.

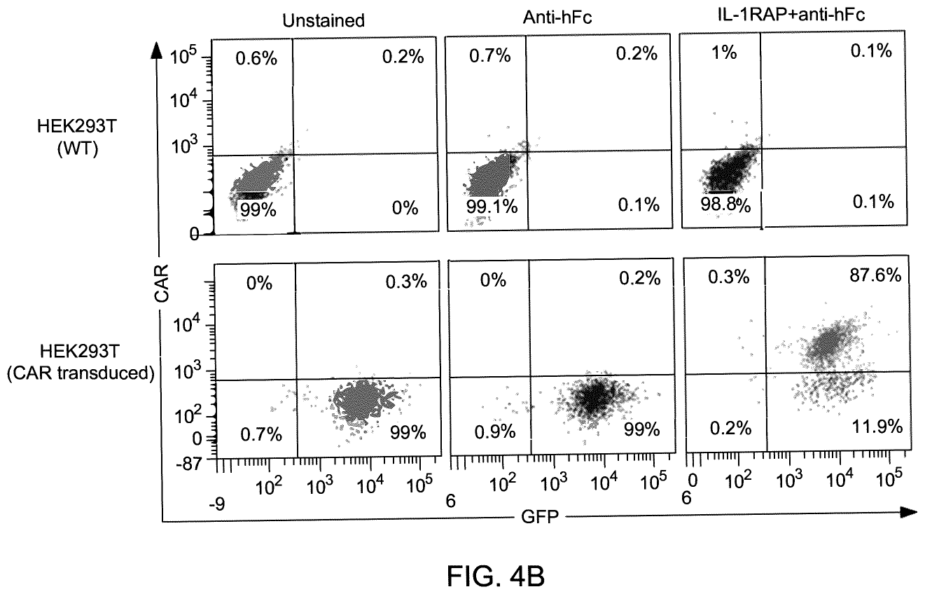

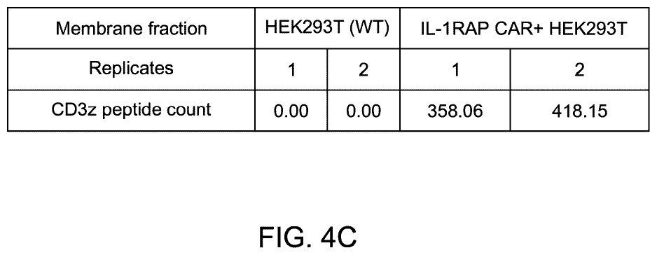

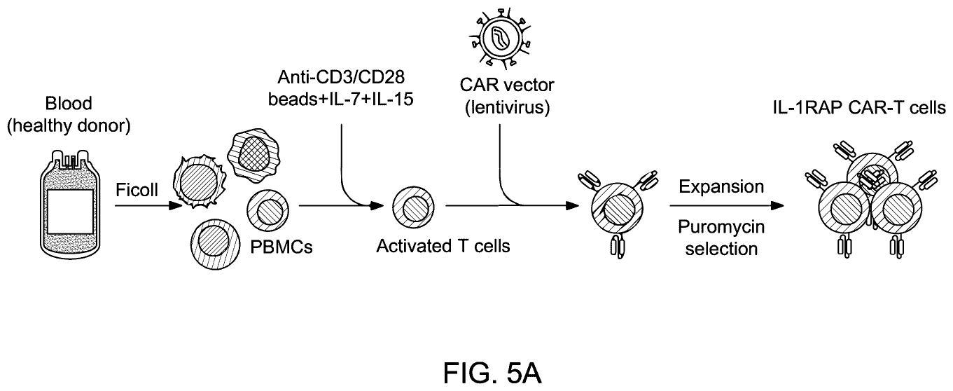

Generation of PTPRD based CAR lentiviral vector: humanized CAR was generated by the fusion of DNA sequence encoding for the extracellular fragment of human PTPRD with the sequences encoding for human CD28 transmembrane region, 4-1BB and CD3z proteins successively. CAR sequence was cloned in lentiviral backbone with GFP and puromycin resistance genes allowing the identification and selection of CAR-T cell (FIG. 4A).

Schematic Representation of the Lentiviral Vector Encoding PTPRD-CD28-4-1BB-CD3ζ CAR Construct. The diagram illustrates the lentiviral vector used to express the PTPRD CAR. The 5′ LTR (long terminal repeat) is truncated to enhance safety. The vector contains the HIV-1 packaging signal (ψ), the Rev-responsive element (RRE), and the central polypurine tract/central termination sequence (cPPT/CTS) for efficient viral packaging and integration. An EF-la core promoter drives the expression of the CAR sequence. The CAR construct includes a Kozak sequence for efficient translation initiation, followed by the coding sequence for a segment from PTPRD, linked to CD28 and CD3ζ signaling domains to provide T cell activation and co-stimulation. Downstream of the CAR, the construct includes CopGFP (green fluorescent protein) linked via a T2A self-cleaving peptide to enable separate expression of GFP and the CAR. A puromycin resistance gene (PuroR), driven by the CMV promoter, is included for selection of successfully transduced cells. The vector also includes a woodchuck post-transcriptional regulatory element (WPRE) to enhance transgene expression and a 3′ LTR (ΔU3) to terminate transcription. In a representative embodiment, the whole CAR DNA length is 1640 bp.

To evaluate the protein expression of IL-IRAP CAR on the cell membrane, HEK293T cells were transduced with CAR vector. Then, after cell fractionation and proteomic analysis by quantitative liquid chromatography mass spectrometry (LC/MS), CD3z peptide (part of CAR protein) has been identified only on CAR transduced HEK293T compared to untransduced HEK293T cells (FIG. 4B and FIG. 4C).

FIGS. 4A-4C describe generation and validation of the IL-1RAP CAR-vector. 4A) Schematic Representation of the Lentiviral Vector Encoding PTPRD-CD28-CD3ζ CAR Construct. The diagram illustrates the lentiviral vector used to express the PTPRD CAR. The 5′ LTR (long terminal repeat) is truncated to enhance safety. The vector contains the HIV-1 packaging signal (ψ), the Rev-responsive element (RRE), and the central polypurine tract/central termination sequence (cPPT/CTS) for efficient viral packaging and integration. An EF-la core promoter drives the expression of the CAR sequence. The CAR construct includes a Kozak sequence for efficient translation initiation, followed by the coding sequence for PTPRD, linked to CD28 and CD3ζ signaling domains to provide T cell activation and co-stimulation. Downstream of the CAR, the construct includes CopGFP (green fluorescent protein) linked via a T2A self-cleaving peptide to enable separate expression of GFP and the CAR. A puromycin resistance gene (PuroR), driven by the CMV promoter, is included for selection of successfully transduced cells. The vector also includes a woodchuck post-transcriptional regulatory element (WPRE) to enhance transgene expression and a 3′ LTR (AU3) to terminate transcription. 4B) CAR detection using recombinant human IL-1RAP in flow cytometry. HEK293T cells were transduced with IL-1RAP CAR vector. Cells were then incubated in presence of recombinant human IL-1RAP conjugated to human Fc. Staining was performed with fluorescent anti-human Fc antibody and analyzed by flow cytometry. Wild type HEK293T cells were used as negative control. 4C) CAR protein expression was evaluated by using label free quantitative liquid chromatography mass spectrometry (LC/MS). Cell fractionation was performed on HEK293T (WT) and transduced with IL-1RAP CAR vector. The results showed the CD3z peptide (part of CAR protein) count in membrane fraction.

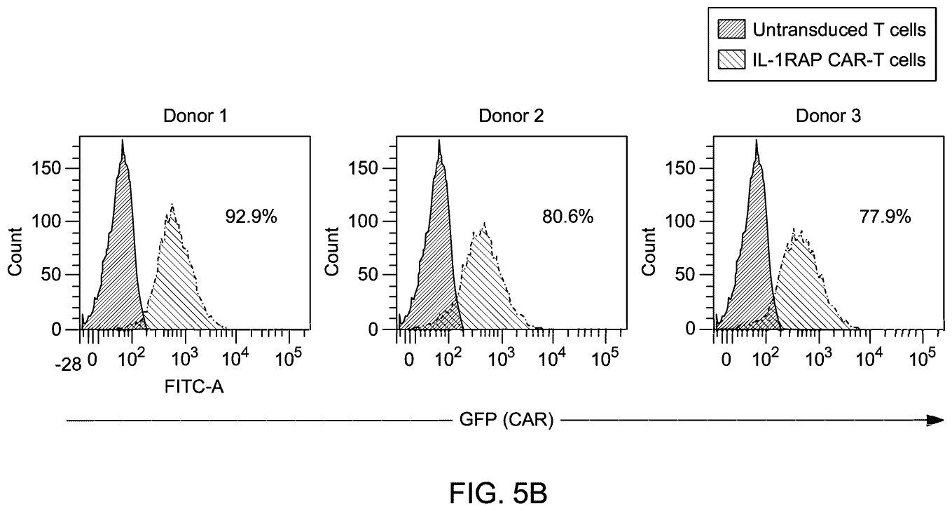

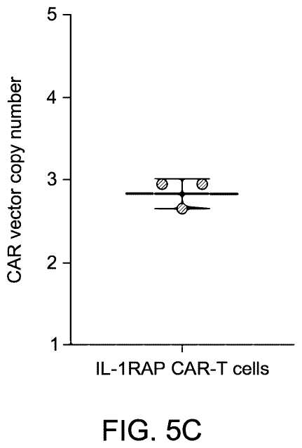

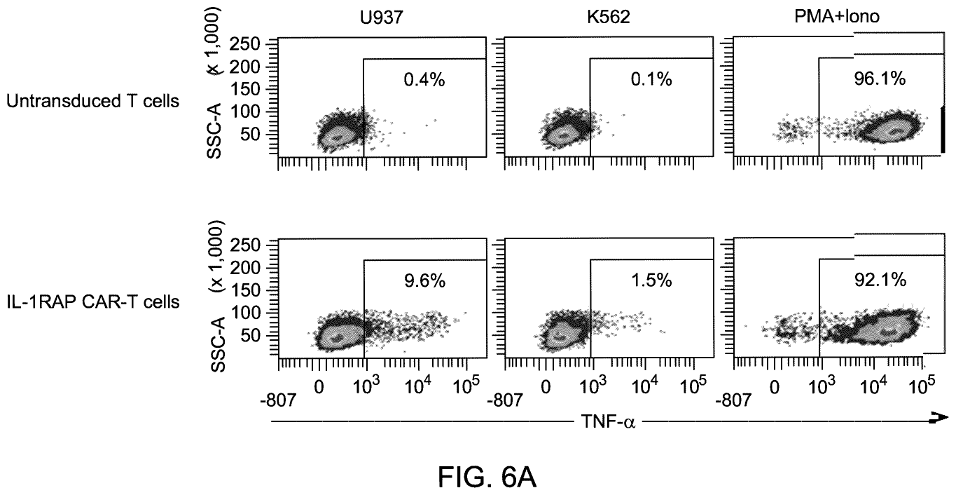

IL-1RAP CAR-T cell are able to secrete pro-inflammatory cytokines and kill AML and breast cancer cells: Healthy donors derived T cells were sorted and activated using CD3/CD28 Dynabeads, then after CAR transduction and selection with resistance to puromycin, the percentage of CAR+ (GFP+) was determined by flow cytometry (FIG. 5A-FIG. 5C). High percentage of GFP+ cells was obtained in three independent experiments. In addition, CAR vector copy number demonstrated the integration in the genome of T cells was determined by PCR (2.84±0.17, n=3).

FIGS. 5A-5C describe generation and qualification of IL-1RAP CAR-T Cells from healthy donors. 5A) Peripheral blood mononuclear cells (PBMCs) were isolated from healthy donor blood using Ficoll density gradient centrifugation. The PBMCs were activated with anti-CD3/CD28 beads in the presence of IL-7 and IL-15 cytokines. Activated T cells were transduced with a lentiviral vector encoding an IL-1RAP CAR construct. Following transduction, CAR-T cells were expanded and selected using puromycin. 5B) Histogram showing GFP (CAR) expression, analyzed by flow cytometry. The percentages of GFP-positive cells, representing CAR-expressing T cells, are shown for three different donors. Gray histograms represent untransduced control cells, and green histograms represent CAR-T cells expressing GFP obtained from three different healthy donors. 5C) CAR vector copy number was determined by PCR on IL-1RAP CAR-T cells using MACS copy check kit.

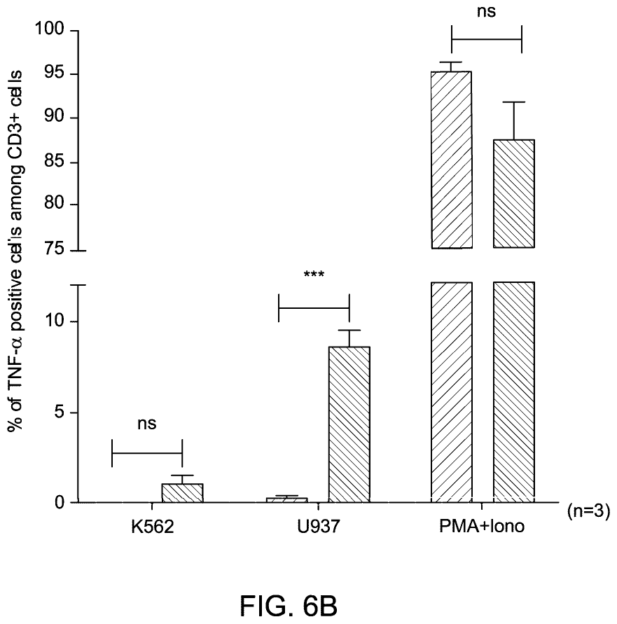

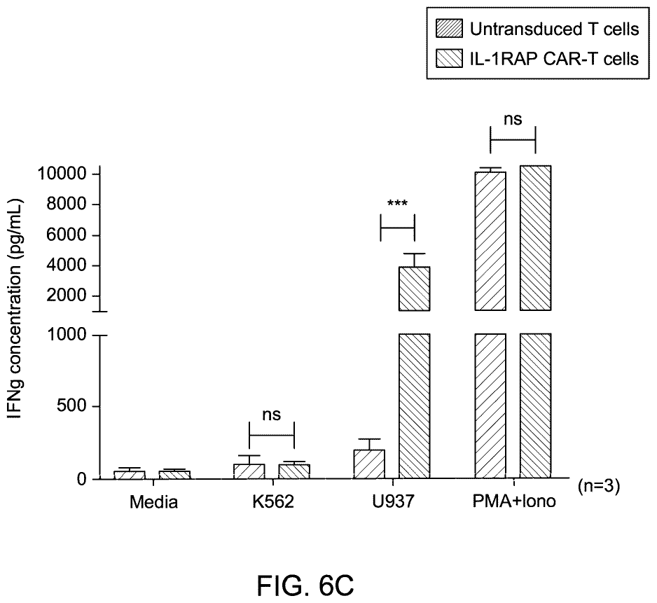

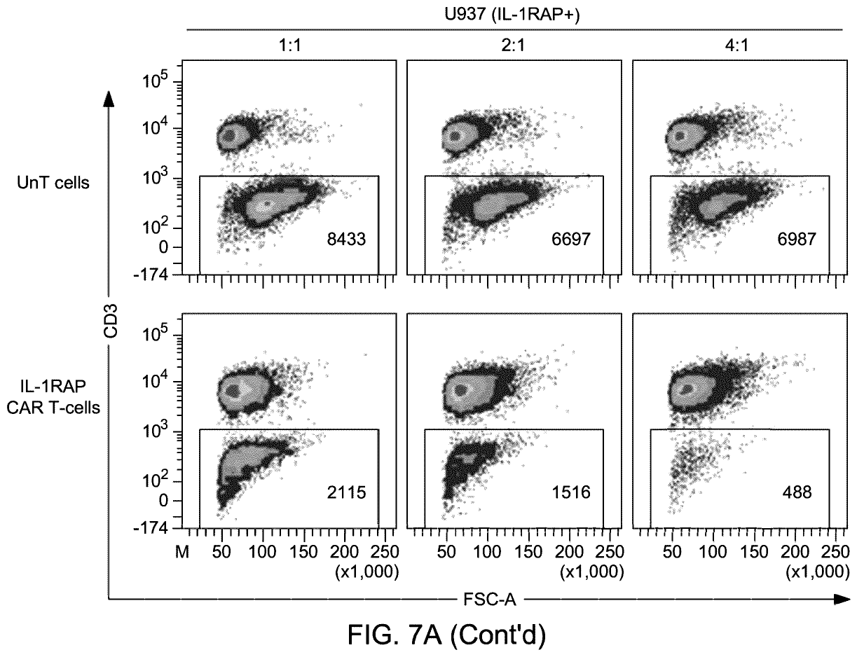

IL-1RAP CAR-T cells were co-cultured with IL-1RAP+ cells (U937 AML cell line) or IL-1RAP-cells (K562 cell line) in order to evaluate their functionality against hematopoietic cancer cells. The results showed a high percentage of TNF-α+ cells with IL-IRAP CAR-T cell compared to untransduced T cells after coculture with U937 (8.6±0.88% vs 0.33±0.11 respectively) (FIG. 6A and FIG. 6B).

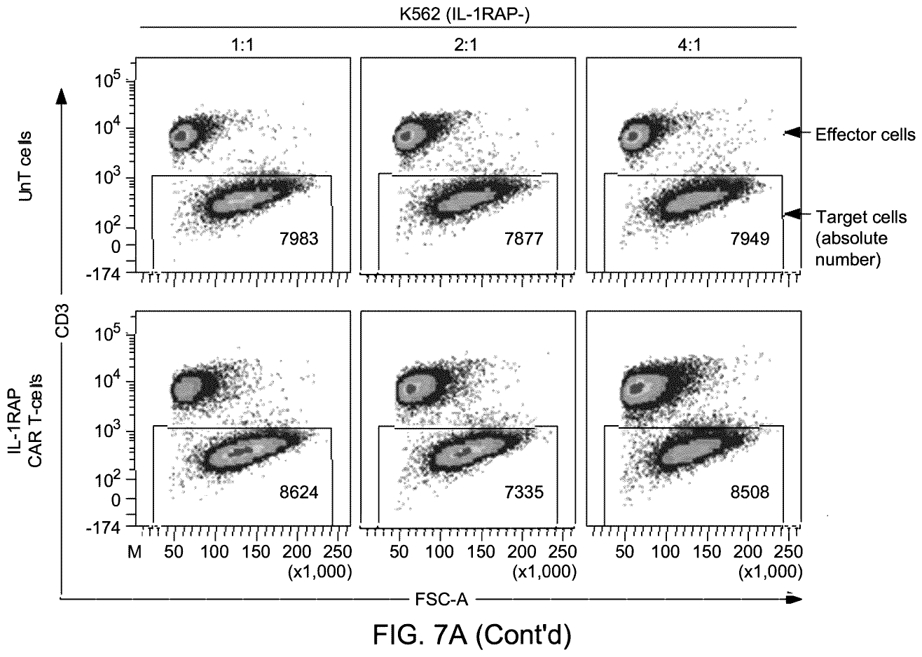

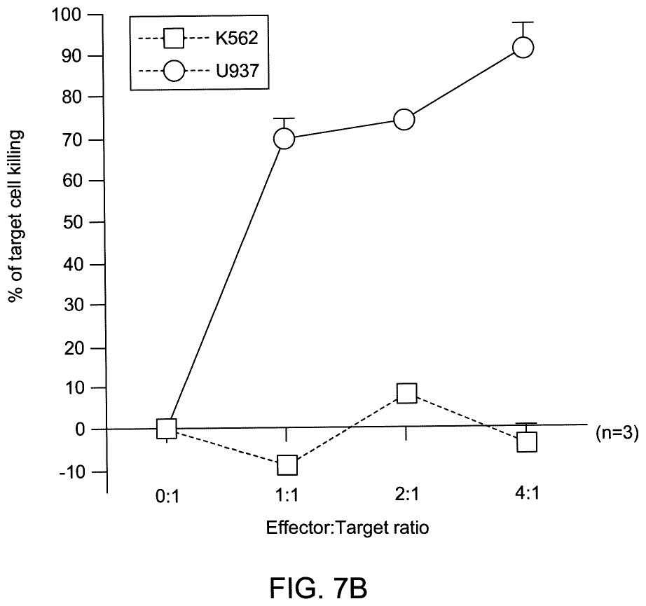

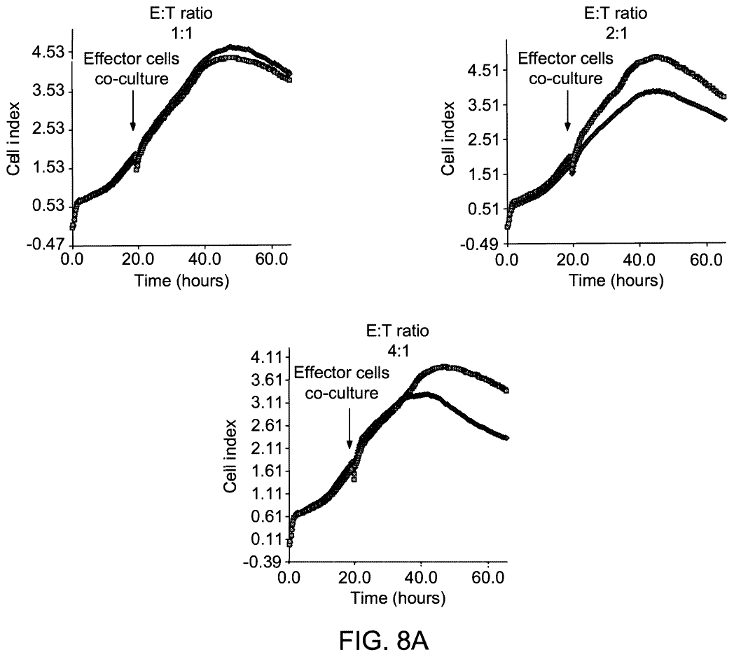

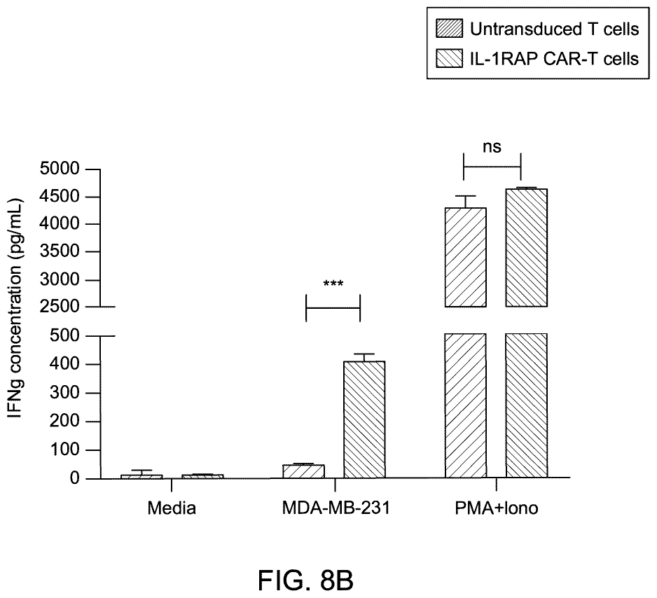

In addition, by ELISA it was demonstrated that IL-1RAP CAR-T cells can release IFNγ after activation and induced the killing of AML cells in effector to target ratio dependent manner (FIG. 6C). Moreover, IL-1RAP CAR-T cells are able to eliminate IL-1RAP+ breast cancer cells (MDA-MB-231) (FIG. 7A and FIG. 7B) and secret IFN-g as shown in (FIG. 8A and FIG. 8B).

FIGS. 8A and 8B show that IL-1RAP CAR-T cells are able to kill breast cancer IL-1RAP+ cells. A) Results of Real-Time Cell Analysis (RTCA) following co-culture of IL-1RAP CAR-T (green lines) or untransduced T cells (blue lines) with MDA-MB-231 breast cancer cells over time. Each graph displays the cell index as a function of time, illustrating the interaction dynamics between the T cells and cancer cells across three different effector: target ratios. B) IFNg concentration measured by ELISA after 24 hours of coculture betwee,Data are representative of the average of triplicates.

FIGS. 7A and 7B describe that IL-1RAP CAR-T cells are able to kill specifically IL-1RAP+ cells. 7A) Flow cytometry analysis of CD3 expression and forward scatter (FSC-A) in co-cultured IL-1RAP CAR-T cells with U937 (IL-1RAP-positive AML cell line) and K562 (IL-1RAP-negative control cell line) at effector-to-target ratios of 1:1, 2:1, and 4:1. The numbers in the plots represent the total event counts of alive target cells. 7B) Quantification of target cell killing by IL-1RAP CAR-T cells. The percentage of target cell killing was calculated for U937 (red squares) and K562 (black squares) at different effector-to-target ratios (1:1, 2:1, and 4:1). Data are representative of three independent experiments (n=3).

FIGS. 6A-6C show activated IL-1RAP CAR-T cell production are able to express pro-inflammatory cytokines. 6A) Intracellular staining of TNF-α following co-culture of CAR-T cells and untransduced T cells with target cells (K562 and U937). After 6 hours of co-culture, cells were stained with CD3 and TNF-α antibodies. 6B) The percentage of TNF-α+ among CD3+ cells was determined. 6C) Dosage of INF gamma concentration in coculture media after 24 hrs determined by ELISA. Data are representative of three independent experiments (n=3). Statistical significance was given as *** by p-values less than <0.0001, untransduced T cells vs. IL-1RAP CAR-T cells.

Two other potential uses of PTPRD need to be investigated. First, PTPRD fused to the human constant region (Fc) could be used to target IL-1RAP+ cells via antibody-dependent cellular cytotoxicity (ADCC). Second, soluble PTPRD could be used as an inhibitor for IL-1b, IL-33, and IL-36 signaling, where IL-IRAP is engaged to induce inflammation in inflammatory diseases. Further development and clinical evaluation of PTPRD-based therapies could significantly improve outcomes for patients with these challenging diseases.

Antibody-dependent cellular cytotoxicity (ADCC) is an advantageous mechanism for tumor targeting for cancer immunotherapy. ADCC may be performed using agents such as PTPRD or monoclonals that specifically recognize tumor antigens like IL-IRAP on cancer cell in combination with effector cells such as NK cells or other CD16+ cells including monocytes/macrophages, NKT cells and γδ T cells that interact with the targeting agent.

Use of PTPRD as an inhibitor of IL-IRAP. In one embodiment, PTPRD can be used as an inhibitor of IL-1RAP, for example, by direct binding to IL-1RAP or binding to associated molecules involved in or required for IL-1RAP functions such as those that modulate cancer or inflammatory diseases or disorders. This interaction suggests that PTPRD can interfere with IL-1RAP's role in inflammation and cancer progression. Inhibition of IL-1RAP functions may comprise administering PTPRD or a domain or fragment thereof to a patient in need of cancer treatment or treatment of an inflammatory disease. PTPRT and is domains or fragments may be administered directly to a patient, for example, intravenously, intramuscularly, subcutaneously, or in situ to a site of inflammation or cancer. Other modes of administration that contact the PTPRT compound with IL-IRAP may also be used. Gene therapy involving the expression of PTPRT or its fragments or domains may be employed.

PRPRT levels may be modulated or reduced using small molecule inhibition or RNA interference. Such reductions by themselves may aggravate cancer or an inflammatory disease for example by increasing cancer growth rate but may be employed in conjunction with chemotherapy or radiation treatment that kills rapidly growing cells. Reduction of PRPRT levels may be used in in vivo or in vitro testing of new anti-cancer drugs or anti-inflammatory agents.

To conclude, the present disclosure represents the first proof of concept of using the PTPRD protein to target IL-1RAP+ cancer cells using CAR-T cells.

Definitions. As used herein, the term “nucleic acid” refers to biomolecules composed of nucleotides. A nucleic acid may be deoxyribonucleic acid (DNA) or ribonucleic acid (RNA).

As used herein, the term “vector” refers to a DNA molecule that acts as a carrier to transfer a DNA sequence to a host cell.

As used herein, the term “protein” or “polypeptide” refers to a sequence of amino acids serving an important biological function.

As used herein, the term “codon” refers to a set of three nucleotide bases in the nucleic acids, DNA or RNA that together code for an amino acid.

As used herein, the term “antigen” refers to a peptide, lipid, foreign particulate matter, polysaccharide or any other similar molecule capable of being recognized by an immune response in the body.

As used herein, the term “immunogen” refers to a peptide, lipid, foreign particulate matter, polysaccharide or any other similar molecule capable of triggering an immune response in the body.

As used herein, the term “antibody” refers to proteins produced by the body in response to an immune reaction particularly, in response to a foreign antigen that has entered the body. Antibodies include those of isotypes IgA, IgE, IgG or IgM.

As used herein, the term “specificity” refers to the ability of an antibody or an antigen to differentiate between other antibodies or antigens.

As used herein, the term “receptor” refers to a protein molecule that binds to specific molecules or ligands, thereby initiating a cellular response.

As used herein, the term “T-cells” refers to lymphocytes that are a part of the immune system and help the body fight infections and diseases.

As used herein, the term “Naive T-cells” refers to immature T cells that have not yet encountered an antigen.

As used herein, the term “Memory T-cells” refers to T cells that persist in the body after an initial infection or vaccination. Memory T cells are responsible for a long-lasting immunity.

As used herein, the term “Regulatory T-cells” refers to T cells that help to maintain an immune system balance by suppressing the activity of other immune cells.

As used herein, the term “CAR” or “chimeric antigen receptor” refers to a receptor created by fusing an extracellular antigen-recognition domain from one molecule linked to intracellular signaling domains of a T-cell.

As used herein, the term “CAR-T cell” refers to T-cells expressing chimeric antigen receptors on their surface.

As used herein, the term “IL-1RAP” refers to a receptor protein that is involved in IL-1, IL-18, IL-33, IL-36 and IL-37 signaling and activates different signaling pathways related to immune system. IL-1RAP is a transmembrane protein with three main domains: an N-terminal extracellular domain, a transmembrane domain, and a C-terminal cytosolic Toll/Interleukin-1 (TIR) domain.

As used herein, the term “IL-1RAP ligand” includes Protein Tyrosine Phosphatase Receptor Type D (PTPRD), segments or variants of PTPRD binding to IL-1RAP, conjugates of PTPRD or its variants or fragments to other molecules.

IL-1RAP acts as a co-receptor and plays a critical role in the signaling pathways of the IL-1 receptor family. It forms functional receptor complexes with several members of this family, partnering with specific primary receptors. Here are its key partners:

IL-1R1 (Interleukin-1 Receptor Type I) which acts to mediate signaling in response to IL-la and IL-1ß cytokines.

IL-1R2 (Interleukin-1 Receptor Type II) which acts as a decoy receptor for IL-1. Unlike IL-1R1, this complex does not transduce signals but instead sequesters IL-1 to reduce inflammatory responses.

IL-33R (ST2) the receptor for IL-33, forming a functional signaling complex, is involved in promoting type 2 immune responses, including mast cell activation, eosinophilia, and tissue repair.

IL-36R (Interleukin-36 Receptor) which acts to mediate signaling in response to IL-36 cytokines (IL-36α, IL-36β, IL-36γ).

IL-18Ra (Interleukin-18 Receptor Alpha) which acts to facilitate immune responses like IFN-γ production, particularly in innate immunity.

As used herein, the term “Protein-Tyrosine Phosphatase Receptor-Type delta” refers to a protein that regulates neurogenesis and is associated with neurodevelopmental disorders.

As used herein, the term “single-chain variable antibody fragment” refers to an antibody fragment formed by the fusion of the variable regions of the heavy and light chains of antibodies.

As used herein, the term “cytokine” refers to proteins that are released by certain cells in response to certain signals and play a role in stimulating or slowing down the immune system.

As used herein, the term “plasmid” refers to a small DNA molecule other than the chromosomal DNA that can replicate on its own.

As used herein, the term “domain” refers to regions of a polypeptide that are folded independently of other regions of the polypeptide and perform a particular function.

As used herein, the term “variant” refers to modified form of a protein that is formed by substitution, deletion or insertion of one or more amino acids into the parent protein chain. A protein variant may have at least 70, 80, 90, 95, 96, 97, 98, 99, or <100% sequence identity or similarity to a reference sequence.

As used herein, the term “extracellular domain” refers to a part of a protein extending outside the cell which is responsible for interacting with other cells, molecules, or the extracellular matrix. For example, antigen binding domain of an antibody.

As used herein, the term “transmembrane domain” refers to a segment of a protein that spans a cell membrane and anchors the protein within the lipid bilayer. These domains help proteins to interact with both intracellular and extracellular environment.

As used herein, the term “cytokine” refers to proteins that are released by certain cells in response to certain signals and play a role in stimulating or slowing down the immune system.

As used herein the term “4-1BB” refers to a co-stimulatory receptor also known as CD137. It is a member of the tumor necrosis factor receptor (TNFR) superfamily and plays a significant role in regulating T cell responses, including enhancing their activation, proliferation, survival, and effector functions.

As used herein the term “CD3z” refers to refers to the & chain of the T-cell receptor complex, a crucial signaling molecule involved in the activation of T cells.

As used herein, the term “subject” refers to a human who requires or may require requires cancer therapy, such as one suspected to have cancer by a medical professional or by one or more previous or partial diagnoses. A subject may be fully histocompatible with a donor or partially histocompatible with a donor. Those skilled in the art may select an appropriate degree of histocompatibility based on matches or mismatches of HLA type-I genes (e.g. HLA-A, HLA-B, or HLA-C) or HLA type II genes (e.g. HLA-DR or HLA-DQ) which increase the risk of graft-versus-host disease.

In some embodiments, amino acid sequences may have 1, 2, 3, 4, 5, 6, 7, 8, 9, 10 or more residue deletions, substitutions, or additions to a known amino acid sequence, such as to the sequences in SEQ ID NOS: 1-5 or the other sequences described herein.

As used herein, the words “a,” “an” and the like generally carry a meaning of “one or more,” unless stated otherwise.

The terms “approximately,” “approximate,” “about,” and similar terms generally refer to ranges that include the identified value within a margin of 20%, 10%, 5%, 2% or 1%, and any values or subranges therebetween.

Numerous modifications and variations of the present disclosure are possible in light of the above teachings. It is therefore to be understood that within the scope of the appended claims, the invention may be practiced otherwise than as specifically described herein.

All publications and patent applications mentioned in this specification are herein incorporated by reference in their entirety to the same extent as if each individual publication or patent application was specifically and individually indicated to be incorporated by reference, especially referenced is disclosure appearing in the same sentence, paragraph, page or section of the specification in which the incorporation by reference appears.

The citation of references herein does not constitute an admission that those references are prior art or have any relevance to the patentability of the technology disclosed herein. Any discussion of the content of references cited is intended merely to provide a general summary of assertions made by the authors of the references and does not constitute an admission as to the accuracy of the content of such references.

1. A T cell comprising a chimeric antigen receptor (CAR) that comprises at least one ligand for Interleukin-1 Receptor Accessory Protein (IL-1RAP).

2. The T cell of claim 1, wherein the ligand for IL-1RAP comprises Protein Tyrosine Phosphatase Receptor Type D (PTPRD) or a fragment thereof that binds to IL-1RAP.

3. The T cell of claim 1, wherein the ligand for IL-1RAP comprises Receptor Protein Tyrosinase Phosphatase delta (PTPRD; SEQ ID NO: 1) or a variant thereof that has at least 95% sequence identity to SEQ ID NO: 1, or a fragment thereof that binds to IL-1RAP.

4. The T cell of claim 1, wherein the ligand for IL-1RAP further comprises a transmembrane domain selected from the group consisting of CD3 chain, CD28, CD4 and CD8a and, optionally, one or more costimulatory domains.

5. The T cell of claim 1, wherein the ligand for IL-1RAP further comprises one or more cytoplasmic costimulatory domains selected from the group consisting of CD32 chain, CD28, 4-1BB (CD137) and OX40 (CD134).

6. The T cell of claim 1 comprising a PTPRD-CD28-4-1BB-CD3 CAR construct and optionally, a Kozak sequence operably linked to or within the construct.

7. The T cell of claim 1, wherein the ligand for IL-1RAP comprises a single-chain variable fragment of an antibody comprising an antigen binding site for IL-1RAP.

8. The T cell of claim 1 that is a CD8+ T cell.

9. The T cell of claim 1 that is a CD4+ T cell.

10. The T cell of claim 1 that is a naïve T cell, a memory T cell, or a regulatory T cell.

11. The T cell of claim 1 that is autologous to a normal cell or cancer cell expressing the IL-1RAP ligand.

12. The T cell of claim 1 that is allogeneic to a normal cell or cancer cell expressing the IL-1RAP ligand, where the T cell shares at least one major HLA antigen with the normal cell or cancer cell expressing the IL-1RAP ligand.

13. The T cell of claim 1 that secretes one or more cytokines that damage or destroy target cells expressing IL-1RAP.

14. A composition comprising the T cell of claim 1 and a medically or pharmaceutically acceptable carrier or excipient, or a culture medium for said T cell.

15. A method for treating cancer or other condition associated with cells expressing IL-1RAP comprising administering the T cell of claim 1 to a subject having the cancer or condition that expresses IL-1RAP.

16. The method of claim 15, wherein the cancer or other condition comprises a solid cancer, a hematological malignancy or blood cancer, or another type of cancer.

17. The method of claim 15, wherein the cancer or other condition comprises chronic myeloblastic leukemia or acute myeloblastic leukemia.

18. The method of claim 15, wherein the cancer or other condition comprises a proliferative disorder, an inflammatory or autoimmune disorder, a neurodegenerative disorder, or a cardiovascular disorder.

19. A nucleic acid or nucleic acid vector encoding the CAR of claim 1 or a component thereof.

20. A method for generating the T cell comprising a chimeric antigen receptor (CAR) that comprises a ligand for Interleukin-1 Receptor Accessory Protein (IL-1RAP) of claim 1 comprising:
isolating PBMCs from blood, activating T cells in the PBMCs with anti-CD3/CD28 in the presence of IL-7 and IL-15 cytokines,
transducing the activated T cells with a vector encoding an IL-1RAP construct, and optionally
expanding the transduced T cells and/or isolating or characterizing the transduced T cells.
